(19) United States
(12) Patent Application Publication
(10) Pub. No.: US 2007/0004041 A1
(43) Pub. Date: Jan. 4, 2007

(54) Heirarchical assembly methods for genome engineering

(75) Inventors: George Church, Brookline, MA (US); Brian M. Baynes, Cambridge, MA (US); Edmund R. Pitcher, Lexington, MA (US)

Correspondence Address: FISH & NEAVE IP GROUP;ROPES & GRAY LLP, ONE INTERNATIONAL PLACE, BOSTON, MA, 02110-2624, US

(73) Assignee: Codon Devices, Inc., Cambridge, MA (US)

(21) Appl. No.: 11/331,927

(22) Filed: Jan. 12, 2006

Related U.S. Application Data
(60) Provisional application No. 60/696,158, filed on Jun. 30, 2005.

(51) Int. Cl. C12N 15/74

(52) U.S. Cl. 435/455; 435/473

(57) ABSTRACT

The present invention provides recombination based methods for assembling nucleic acids. In certain aspects the present invention provides hierarchical assembly methods for producing genome sized polynucleotide constructs. The methods may be used for assembling large polynucleotide constructs, for synthesizing synthetic genomes, or for introducing a plurality of nucleotide changes throughout the genome of an organism. In another aspect, the invention provides cells having increased genomic stability. For example, cells comprising alterations in at least a substantial portion of the transposons in the genome are provided.

### RELATED APPLICATIONS

This application claims the benefit of priority to U.S. Provisional Patent Application No. 60/696,158, filed Jun. 30, 2005, which is hereby incorporated by reference in its entirety.

### BACKGROUND

Cells have a number of well-established uses in molecular biology. For example, cells are commonly used as hosts for manipulating DNA in processes such as transformation and recombination. Cells are also used for expression of recombinant proteins encoded by DNA transformed/transfected or otherwise introduced into the cells. Some types of cells are also used as progenitors for generation of transgenic animals and plants. Although all of these processes are now routine, in general, the genomes of the cells used in these processes have evolved little from the genomes of natural cells, and particularly not toward acquisition of new or improved properties for use in the above processes.

The traditional approaches to modification of cellular genomes have various limitations. For example, it is possible to make specified predetermined changes to the genome, however, each change requires time intensive procedures to produce the desired modifications. Alternatively, it is possible to make large numbers of genome modifications using non-specific mutagenesis techniques. This approach allows the researcher to make genome wide modifications to an organism but permit little to no control over the types of modifications which are made. Accordingly, there is a need for techniques that would permit rapid, planned, genome-wide engineering of an organism. Such techniques would permit a researcher to develop cells with improved properties such as commercial utility and/or enhanced safety. For example, genome wide engineering may be used to improve a cell's capacity to express a recombinant protein which might require modification in any or all of a substantial number of genes having roles in transcription, translation, posttranslational modification, secretion or proteolytic degradation, among others. Additionally, the potential escape of genetically engineered organisms into the environment and the integration of their genomes and capabilities with wild-type organisms is widely viewed as an environmental threat. The ability to create genome wide modifications to an organism's genome would permit the genetic isolation of an organism from wild-type organisms and thereby reduce safety concerns.

### SUMMARY OF THE INVENTION

The invention relates to methods for producing large nucleic acid constructs, such as genome sized constructs, and organisms having modified, partially synthetic, and/or fully synthetic genomes such as prokaryotes, archaebacteria, fungi, yeasts, animals, and plants. For example, the invention permits synthesis of modified, partially synthetic, and/or fully synthetic genomes having a plurality of predetermined and specified alterations throughout the genome. Such modified, partially synthetic, and/or fully synthetic genomes may provide improved properties to a host cell such as commercial utility, genome stability, or increased safety. Synthetic DNA is DNA originating at least in part from the extracellular chemical synthesis of a sequence of nucleotide bases, as opposed to replication of a template sequence, and permits, for example, a portion of the organism's genome to be redesigned.

The methods described herein permit purposeful or experimental alteration of expression in various ways. It permits the genetic engineer to modulate level of expression in a cell of native form proteins by modifying codons within genes using the redundancy of the genetic code so as to exploit codon bias of particular cell types and species. It permits alteration of open reading frames so as to select from among variants of a protein or proteins. It also permits construction of organisms having an altered genetic code including, for example: organisms which are adapted stably to incorporate non-natural amino acids into expressed proteins so as to enable production of a new class of protein structures; and organisms which cannot receive, cannot donate, can neither receive nor donate, or cannot exchange protein-encoding genetic information effectively with any wild type organism and therefore cannot exchange traits derived from proteins with wild type organisms. Organisms having a modified genetic code may be constructed by making predetermined, genome wide nucleotide alterations or by constructing an entire genome from synthetic polynucleotide constructs and/or from polynucleotide constructs having modified genomic sequences.

In one aspect, the invention provides a cell having increased genetically stability comprising mutations in at least a substantial portion of the open reading frames, or regulatory regions, of the transposase genes in the genome, wherein said mutations significantly reduce or prevent production of functional transposase, thereby improving genetic stability of said cell.

In various embodiments, the cell having increased genetic stability may comprise one or more of the following types of mutations: point mutations; mutations that introduce at least one, two or more stop codons into the open reading frames of the transposase genes; missense mutation in the open reading frames of the transposase genes; at least one mutation in a conserved and/or functionally important region of a transposase; mutations that are located in the open reading frame of the transposase in proximity to the translational start site; mutations in the transcriptional control sequences of the transposase genes; mutations in the translational control sequences of the transposase genes; mutations that are located in inverted repeat sequences of the transposase genes; and/or mutations that cause translation termination near the N-terminus of the transposases (e.g., within 50 amino acids, 25 amino acids, 10 amino acids, 5 amino acids, or less, of the N-terminus). In certain embodiments, a cell having increased genetic stability may have two or more mutations in at least a portion of the open reading frames, or regulatory regions, of the transposase genes. In one embodiment, a cell having increased genetic stability comprises at least one mutation in all of the open reading frames, or regulatory regions, of the transposase genes in the genome.

In an exemplary embodiment, the mutations introduced into the genome to produce the cell having increased genetic stability do not substantially change the genome size or spacing.

In various embodiments, the cell having increased genetic stability may be, for example, a prokaryotic cell, such as a bacterial cell. In an exemplary embodiment, the cell is an E. coli cell.

In another aspect, the invention provides a cell comprising a partially or wholly synthetic genome wherein at least a substantial portion of the open reading frames, or regulatory regions, of the transposase genes in the genome are mutated to significantly reduce or prevent translation of functional transposase, thereby improving genetic stability of said cell.

In another aspect, the invention provides a method for assembling a polynucleotide product, comprising:

i) providing a plurality of cells comprising a plurality of polynucleotide constructs, wherein a portion of the plurality of polynucleotide constructs comprise sequence encoding a first selectable marker and a portion of the plurality of polynucleotide constructs comprise sequence encoding a second selectable marker;

ii) conducting pairwise conjugations by mixing pairs of cells, wherein each pair comprises a cell having at least one polynucleotide construct encoding said first selectable marker and a cell having at least one polynucleotide construct encoding said second selectable marker;

iii) selecting cells comprising at least portions of the polynucleotide constructs from both cells involved in the pairwise mixing that have been assembled in a desired manner by selecting cells comprising one of the first or second selectable markers; and

iv) reiteratively repeating said steps ii) and iii) to form a desired polynucleotide product.

In certain embodiments, the method further comprises introducing the plurality of polynucleotide constructs into the cells, for example, by a method such as electroporation.

In certain embodiments, the method may utilize cells expressing traF, traG, and traJ, optionally under the control of a regulatable promoter.

In certain embodiments, the polynucleotide constructs assemble in a desired manner by integrating into the host cell genome by homologous recombination, site-specific recombination, or combinations thereof. When assembly involves homologous recombination, the host cells may express a recombinase such as, for example, recE and recT from E. coli or the Redα and Redβ proteins from lambda. Expression of the recombinase may optionally be under the control of a regulatable promoter and/or may optionally be overexpressed in the host cell.

In certain embodiments, the polynucleotide constructs may be contained on an extrachromosomal plasmid or an artificial chromosome.

In certain embodiments, at least one terminal sequence of each polynucleotide construct is homologous with the terminal sequences of another polynucleotide construct. Such homologous terminal regions may be at least about 20, 50, or more nucleotides in length.

In certain embodiments, the cells may be bacterial cells, such as, for example, E. coli.

In certain embodiments, the first and/or second selectable markers confer a cell survival advantage in a defined medium. For example, the first and second selectable markers may be kanamycin resistance and chloramphenicol resistance. In certain embodiments, the first and/or second selectable markers confer a detectable phenotypic change to the cells.

In certain embodiments, one or more polynucleotide constructs may comprise: (i) a meganuclease cleavage site near one terminus or near both termini, (ii) an origin of replication near one terminus, (iii) an oriT site near one terminus, and/or (iv) an oriT site near one terminus and an origin of replication near the other terminus.

In certain embodiments, polynucleotide constructs comprising sequence encoding a said first selectable marker further comprise a first oriT, a first origin of replication, and one or more cleavage sites for a first meganuclease and polynucleotide constructs comprising sequence encoding a said second selectable marker further comprise a second oriT, a second origin of replication, and one or more cleavage sites for a second meganuclease. The first and second oriTs may be, for example, ColE1 oriT and incBCD oriT. The first and second origins of replication may be, for example, IncX R6K oriγ and IncPα oriV. The first and second meganuclease cleavage sites may be cleavage sites recognized by one of the following meganucleases: I-SceI, I-DmoI, I-CreI, or I-DreI-3.

In certain embodiments, the plurality of cells may comprise sequence encoding a negative selectable marker.

In certain embodiments, polynucleotide constructs comprising sequence encoding a said first selectable marker are introduced into a plurality of cells comprising sequence encoding a first negative selectable marker and polynucleotide constructs comprising sequence encoding a said second selectable marker are introduced into a plurality of cells comprising sequence encoding a second negative selectable marker.

In certain embodiments, the polynucleotide product may be a genome.

In another aspect, the invention provides a method for remapping all essential portions of a genome of a cell, the method comprising assembling a genome by a method comprising:

i) providing a plurality of cells comprising a plurality of polynucleotide constructs, wherein a portion of the plurality of polynucleotide constructs comprise sequence encoding a first selectable marker and a portion of the plurality of polynucleotide constructs comprise sequence encoding a second selectable marker;

ii) conducting pairwise conjugations by mixing pairs of cells, wherein each pair comprises a cell having at least one polynucleotide construct encoding said first selectable marker and a cell having at least one polynucleotide construct encoding said second selectable marker;

iii) selecting cells comprising at least portions of the polynucleotide constructs from both cells involved in the pairwise mixing that have been assembled in a desired manner by selecting cells comprising one of the first or second selectable markers; and

iv) reiteratively repeating said steps ii) and iii) to form a desired polynucleotide product,

wherein the plurality of polynucleotide constructs together comprise all portions of a genome that are essential for survival of said cell, and wherein said plurality of polynucleotide constructs comprise sequences having at least one codon remapped throughout the genome.

In certain embodiments, the genome is the genome of the host cell used for the assembly method.

In another aspect, the invention provides a method of reducing or preventing translation of functional transposase in a cell, the method comprising

assembling a genome by a method comprising:

i) providing a plurality of cells comprising a plurality of polynucleotide constructs, wherein a portion of the plurality of polynucleotide constructs comprise sequence encoding a first selectable marker and a portion of the plurality of polynucleotide constructs comprise sequence encoding a second selectable marker;

ii) conducting pairwise conjugations by mixing pairs of cells, wherein each pair comprises a cell having at least one polynucleotide construct encoding said first selectable marker and a cell having at least one polynucleotide construct encoding said second selectable marker;

iii) selecting cells comprising at least portions of the polynucleotide constructs from both cells involved in the pairwise mixing that have been assembled in a desired manner by selecting cells comprising one of the first or second selectable markers; and

iv) reiteratively repeating said steps ii) and iii) to form a desired polynucleotide product,

wherein said plurality of polynucleotide constructs together comprise a modification in at least a substantial portion of open reading frames or regulatory regions of transposase genes, and wherein said modification causes a reduction in or prevents translation of functional transposase in a cell.

In certain embodiments, at least a portion of the polynucleotide constructs are constructed from synthetic DNA.

In certain embodiments, the method further comprises excising a plurality of polynucleotide sequence segments from a naturally-occurring genome and modifying the sequences of said segments thereby forming said plurality of polynucleotide constructs. Optionally, at least a portion of the excised segments may be modified in parallel.

In another aspect, the invention provides a method for introducing a plurality of predetermined nucleotide changes throughout a polynucleotide product, comprising:

modifying one or more nucleotides on each of a plurality of polynucleotide segments from a genome to form a plurality of polynucleotide constructs; and

incorporating said plurality of polynucleotide constructs into said genome thereby introducing a plurality of nucleotide changes throughout said polynucleotide product.

In certain embodiments, the plurality of polynucleotide segments are each at least about 50 kilobases in length, 100 kilobases in length, or longer.

In certain embodiments, at least a portion of said polynucleotide segments may be modified in parallel.

In certain embodiments, the polynucleotide product is a genome, such as a bacterial genome. In an exemplary embodiment, the genome is an E. coli genome.

In certain embodiments, the step of incorporating said plurality of polynucleotide constructs into said genome is conducted serially or hierarchically.

In certain embodiments, the method further comprises excising a plurality of polynucleotide segments from a genome. The polynucleotide segments may beexcised from the genome using site-specific recombination, such as, for example, site-specific recombination mediated by FLP/FRT or Cre/LoxP.

In certain embodiments, the method further comprises:

introducing site-specific recombination sequences into the genome at locations flanking one or more polynucleotide segments to be excised from the genome; and

exposing the genome to a site-specific recombinase thereby inducing intramolecular recombination between the site-specific recombination sequences and excising one or more polynucletide segments from the genome.

In certain embodiments, a host cell containing the genome comprises a polynucleotide sequence encoding the site-specific recombinase under the control of a regulatable promoter.

In certain embodiments, the method further comprises:

introducing a conditional origin of replication into the genome between the site-specific recombination sequences; and

exposing the excised polynucleotide segments to a replication initiation protein thereby amplifying copy number of the excised polynucleotide segments.

In certain embodiments, a host cell containing the genome comprises a polynucleotide sequence encoding said replication initiation protein under the control of a regulatable promoter.

In certain embodiments, the site-specific recombination sequences may be LoxP sites that are introduced into the genome in the same orientation and the site-specific recombinase is Cre.

In certain embodiments, the conditional origin of replication is R6K Oriγ.

In certain embodiments, the replication initiation protein is π.

In certain embodiments, the polynucleotide segments are modified by PCR mutagenesis, site-specific mutagenesis, site-specific recombination, or homologous recombination.

In certain embodiments, the method further comprises:

introducing said plurality of polynucleotide constructs into a plurality of cells, wherein the sequences of the plurality of polynucleotide constructs together comprise the sequence of the polynucleotide construct, and wherein each polynucleotide construct comprises sequence encoding at least one of a first or second selectable marker, thereby forming a first set of transfected cells;

mixing pairwise cells from the first set of transfected cells, wherein each pair comprises a cell having polynucleotide construct encoding said first selectable marker and a cell having a polynucleotide construct encoding said second selectable marker, thereby forming a second set of transfected cells;

reiteratively repeating said mixing step, wherein the second set of transfected cells becomes the first set of transfected cells for the next round of pairwise mixing, thereby incorporating said plurality of polynucleotide constructs into said polynucleotide product and introducing a plurality of nucleotide changes throughout said polynucleotide product.

In certain embodiments, the pairwise mixing of cells involves conjugation and transfer of a modified polynucleotide segment from one cell to another cell.

In certain embodiments, the polynucleotide constructs integrate into the polynucleotide product by homologous recombination.

In certain embodiments, the polynucleotide product is a genome.

In certain embodiments, the polynucleotide constructs comprising sequence encoding a said first selectable marker further comprise a first oriT, a first origin of replication, and one or more cleavage sites for a first meganuclease and the polynucleotide constructs comprising sequence encoding a said second selectable marker further comprise a second oriT, a second origin of replication, and one or more cleavage sites for a second meganuclease.

In certain embodiments, the polynucleotide constructs comprising sequence encoding a said first selectable marker are introduced into a plurality of cells comprising sequence encoding a first negative selectable marker and the polynucleotide constructs comprising sequence encoding a said second selectable marker are introduced into a plurality of cells comprising sequence encoding a second negative selectable marker.

In certain embodiments, the method may be used to codon remap all essential portions of a genome, such as, for example, the genome of one of said plurality of cells.

In certain embodiments, at least about 100, 300, 500, 1,000 or more predetermined nucleotide changes are introduced throughout the polynucleotide product. In exemplary embodiments, all or substantially all of the nucleotide changes are non-contiguous.

In certain embodiments, the predetermined nucleotide changes comprise changing all occurrences of at least a first codon in a given sequence to a second codon that is degenerate to the first codon. In an exemplary embodiment, the first and second codons are stop codons. In another embodiment, the predetermined nucleotide changes comprise changing one or more codons to the first codon wherein the codons that are changed are not degenerate to the first codon.

In certain embodiments, the genome is contained in a cell that does not express a wild-type tRNA that recognizes the first codon. Optionally, the cell contains at least one gene encoding a first modified tRNA that recognizes said first codon but is charged with an amino acid not normally encoded by the first codon.

In an exemplary embodiment, the genome is contained in a cell that does not express a wild-type release factor that recognizes the first codon.

In another aspect, the invention provides a cell comprising a tRNA that inserts an amino acid in response to a stop codon wherein the cell does not express at least one release factor that binds to said stop codon. In an exemplary embodiment, the invention provides a cell comprising a codon remapped genome wherein all, or substantially all, original occurrences of a first stop codon in the genome have been replaced with a second stop codon and wherein the cell does not express (or has substantially reduced expression of) at least one release factor that binds to said first stop codon. The cell may further comprise a tRNA that inserts a natural or non-natural amino acid in response to said first stop codon. The cell may further comprise sequences that comprise one or more occurrences of first stop codon, in locations different from the original occurrences of the first stop codon, where it is desirable to insert the amino acid charged by the tRNA that recognizes the stop codon. The sequences containing the new occurrences of the first stop codon may be in the genome of the host cell or may be maintained on an extrachromosal sequences, such as, for example, a plamid.

In another aspect, the invention provides a method for assembling a polynucleotide product, comprising:

i) providing a plurality of cells comprising a plurality of polynucleotide constructs, wherein a portion of the plurality of polynucleotide constructs comprise sequence X and a portion of the plurality of polynucleotide constructs comprise sequence Y.

wherein X and Y each independently comprise one or more of the following: (i) a selectable marker, (ii) a counterselectable marker, or (iii) non-marker sequence, and wherein at least one of X and Y comprises at least one of the following: (i) a selectable marker, or (ii) a counterselectable marker;

ii) conducting pairwise conjugations by mixing pairs of cells, wherein each pair comprises a cell having at least one polynucleotide construct comprising sequence X and a cell having at least one polynucleotide construct encoding sequence Y;

iii) selecting cells comprising at least portions of the polynucleotide constructs from both cells involved in the pairwise mixing that have been assembled in a desired manner by selecting cells for the presence and/or absence of sequence X and/or Y; and

iv) reiteratively repeating said steps ii) and iii) to form a desired polynucleotide product.

In certain embodiments, introduction of a polynucleotide having sequence Y into a cell comprising a polynucleotide construct having sequence X results in destruction of at least a portion of sequence X.

In certain embodiments, sequence X comprises a selectable marker and a counterselectable marker and introduction of a polynucleotide construct having sequence Y into a cell comprising a polynucleotide construct having sequence X results in destruction of the counterselectable marker of sequence X or destruction of the selectable marker and counterselectable marker of sequence X. In certain such embodiments, following pairwise mixing, cells are selected for the absence of the counterselectable marker of sequence X.

In certain embodiments, sequence Y comprises a non marker sequence.

In other embodiments, sequence Y comprises a selectable marker different from that of sequence X. In such embodiments, following pairwise mixing, cells may be selected for (i) the absence of the counterselectable marker of sequence X, (ii) the presence of the selectable marker of sequence Y, or (iii) the absence of the counterselectable marker of sequence X and the presence of the selectable marker of sequence Y.

In another embodiment, sequence Y comprises a selectable marker and a counterselectable marker that are different from the selectable marker and counterselectable markers of sequence X. In such embodiments, following pairwise mixing, cells may be selected for (i) the absence of the counterselectable marker of sequence X, (ii) the presence of the selectable marker and/or counterselectable marker of sequence Y, or (iii) the absence of the counterselectable marker of sequence X and the presence of the selectable marker and/or coutnerselectable marker of sequence Y.

In another aspect, the invention provides a method of assembling a polynucleotide product comprising:

(a) selecting a double stranded initiating polynucleotide construct;

(b) contacting said initiating polynucleotide construct with a next polynucleotide construct in the presence of a recombination system, wherein said next polynucleotide construct is double stranded and a terminal region of said next polynucleotide construct comprises substantial sequence homology with a terminal region of said initiating polynucleotide construct, and wherein said next polynucleotide construct is joined to said initiating polynucleotide construct by homologous recombination at the terminal regions having substantial sequence homology; and

(c) repeating (b) to sequentially add additional double stranded polynucleotide constructs to the extended initiating polynucleotide construct, whereby said polynucleotide product is synthesized.

In certain embodiments, the recombination system is in a cell. In certain embodiments, said contacting is conducted in a cell. In certain embodiments, said recombination system is extracellular. In certain embodiments, said initiating polynucleotide construct, said next polynucleotide construct, or both, comprises a selectable, a counterselectable marker, or both. In certain embodiments, said initiating polynucleotide construct and said next polynucleotide construct comprise different selectable and/or counterselectable markers. In certain embodiments, said polynucleotide product is at least 5 Mbps. In certain embodiments, said polynucleotide product is a genome. In certain embodiments, said assembly is carried out serially, hierarchically, or a combination thereof. In certain embodiments, said polynucleotide constructs are introduced into the genome of the cell by homologous recombination. In certain embodiments, said polynucleotide product is assembled using an extrachromosomal scaffold. In certain embodiments, said polynucleotide product is assembled using a scaffold nucleic acid. In certain embodiments, the initiating polynucleotide construct and/or the next polynucleotide construct are excised from a genome and optionally modified. In certain embodiments, the initiating polynucleotide construct and/or the next polynucleotide construct are at least partially synthetic.

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims. The claims provided below are hereby incorporated into this section by reference.

### BRIEF DESCRIPTION OF THE FIGURES

FIG. 1 shows a schematic of a sequential assembly procedure.

FIG. 2 shows a schematic of a hierarchical assembly procedure.

FIGS. 3A-D show an illustration of a method for hierarchical assembly procedure. FIG. 3A shows the starting materials including a cell with an initial genome (empty) and variety of polynucleotide constructs (hatched; labeled A, B, C, and D). FIG. 4B illustrates intermediates having a single polynucleotide construct integrated into the host genome. FIG. 3C shows the product of conjugation followed by homologous recombination to produce intermediates having two synthetic polynucleotide constructs integrated into the genome. FIG. 3D illustrates the final modified genome produced by further rounds of conjugation and homologous recombination.

FIG. 4 shows a schematic of another hierarchical assembly procedure.

FIGS. 5A-H show an illustration of a method for excising large segments from a polynucleotide sequence, such as a genome. FIG. 5A shows a genome and the location for incorporation of the constructs shown in FIG. 5B. Also illustrated is Cre gene under the control of a regulatable promoter. FIG. 5B illustrates three polynucleotide constructs that may be incorporated into a genome for directed excision of a segment of the genome. FIG. 5C illustrates the genome having the constructs shown in FIG. 5B incorporated therein. FIG. 5D illustrates homologous recombination between the two loxP sites resulting in excision of the genome segment between the loxP sites. FIG. 5E illustrates the circular polynucleotide segment excised from the genome. FIG. 5F illustrates the polynucleotide segment excised from the genome after cleavage with SceI. FIG. 5G illustrates reintroduction of the modified polynucleotide segment into the genome by homologous recombination. FIG. 5H illustrates a genome having the excised and modified polynucleotide segment incorporated therein.

### DETAILED DESCRIPTION OF THE INVENTION

1. Definitions

The term “codon remapping” refers to modifying the codon content of a nucleic acid sequence without modifying the sequence of the polypeptide encoded by the nucleic acid. In certain embodiments, the term is meant to encompass “codon optimization” wherein the codon content of the nucleic acid sequence is modified to enhance expression in a particular cell type. In other embodiments, the term is meant to encompass “codon normalization” wherein the codon content of two or more nucleic acid sequences are modified to minimize any possible differences in protein expression that may arise due to the differences in codon usage between the sequences. In still other embodiments, the term is meant to encompass modifying the codon content of a nucleic acid sequence as a means to control the level of expression of a protein (e.g., either increases or decrease the level of expression). Codon remapping may be achieved by replacing at least one codon in the “wild-type sequence” with a different codon encoding the same amino acid that is used at a higher or lower frequency in a given cell type. In other embodiments, the term is meant to encompass “codon reassignment” wherein a cell comprises a modified tRNA and/or tRNA synthetase so that the cell inserts an amino acid in response to a codon that is different than the amino acid inserted by a wild-type cell. Furthermore, nucleotide sequences in the cell have been correspondingly modified so that polypeptide sequences encoded by the cell comprising the modified tRNA and/or tRNA synthetase are the same as the polypeptide produced in a wild-type cell.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “degenerate codons” refers to two or more codons that encode for the same amino acid or a translational stop. For example, UUA, UUG, CUU, CUC, CUA and CUG are degenerate codons that encode for the amino acid leucine. Similarly, UAA, UAG and UGA are degenerate codons that signal a translational stop (e.g., “stop codons”).

The term “essential gene” refers to a nucleic acid that encodes a polypeptide or RNA whose function is required for survival, growth, and/or cell division.

The term “frt site” refers to a nucleotide sequence at which the product of the FLP gene of the yeast 2 μm plasmid, FLP recombinase, can catalyze a site-specific recombination.

The term “gene” refers to a nucleic acid comprising an open reading frame encoding a polypeptide having exon sequences and optionally protein non-coding sequences, such as intron or intergenic sequences. The term “intron” refers to a DNA sequence present in a given gene which is not translated into protein and is generally found between exons.

The term “genome” refers to the whole hereditary information of an organism that is encoded in the DNA (or RNA for certain viral species) including both coding and non-coding sequences. In various embodiments, the term may include the chromosomal DNA of an organism and/or DNA that is contained in an organelle such as, for example, the mitochondria or chloroplasts. The term “mitochondrial genome” refers to the genetic material contained in the mitochondria and the term “chloroplast genome” refers to the genetic material contained in the chloroplast.

The term “lox site” refers to a nucleotide sequence at which the product of the cre gene of bacteriophage P1, Cre recombinase, can catalyze a site-specific recombination. A variety of lox sites are known to the art including the naturally occurring loxP (the sequence found in the P1 genome), loxB, loxL and loxR (these are found in the E. coli chromosome) as well as a number of mutant or variant lox sites such as loxP511, lox.DELTA.86, loxΔ117, loxC2, loxP2, loxP3, loxP23, loxS, and loxH.

The terms “modulation” or “modulates”, when used in reference to expression of a polypeptide, refers to the capacity to either up regulate gene expression (e.g., increase expression by activation or stimulation) or down regulate gene expression (e.g., decrease expression by inhibition or suppression).

The term “naturally-occurring”, as applied to an object, refers to the fact that an object may be found in nature. For example, a polypeptide or polynucleotide sequence that is present in an organism (including bacteria) that may be isolated from a source in nature and which has not been intentionally modified by man in the laboratory is naturally-occurring or “wild type”.

As used herein, the term “nucleic acid” refers to polynucleotides such as deoxyribonucleic acid (DNA), and, where appropriate, ribonucleic acid (RNA). The term should also be understood to include analogs of either RNA or DNA made from nucleotide analogs (including analogs with respect to the base and/or the backbone, for example, peptide nucleic acids, locked nucleic acids, mannitol nucleic acids etc.), and, as applicable to the embodiment being described, single-stranded (such as sense or antisense), double-stranded or higher order polynucleotides.

As used herein, the term “origin of replication” refers to an origin of replication that is functional in a broad range of prokaryotic host cells (i.e., a normal or non-conditional origin of replication such as the ColE1 origin and its derivatives). A “conditional origin of replication” refers to an origin of replication that requires the presence of a functional trans-acting factor (e.g., a replication factor) in a prokaryotic host cell.

The term “polypeptide”, and the terms “protein” and “peptide” which are used interchangeably herein, refers to a polymer of amino acids, including, for example, gene products, naturally-occurring proteins, homologs, orthologs, paralogs, fragments, and other equivalents, variants and analogs of the foregoing.

The term “restriction endonuclease recognition site” refers to a nucleic acid sequence capable of binding one or more restriction endonucleases. The term “restriction endonuclease cleavage site” refers to a nucleic acid sequence that is cleaved by one or more restriction endonucleases. For a given enzyme, the restriction endonuclease recognition and cleavage sites may be the same or different. Restriction enzymes include, but are not limited to, type I enzymes, type II enzymes, type IIS enzymes, type III enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts R J et al. (2005) REBASE—restriction enzymes and DNA methyltransferases. Nucleic Acids Res. 33 Database Issue: D230-2).

The term “selectable marker” refers to a polynucleotide sequence encoding a gene product that alters the ability of a cell harboring the polynucleotide sequence to grow or survive in a given growth environment relative to a similar cell lacking the selectable marker. Such a marker may be a positive or negative selectable marker. For example, a positive selectable marker (e.g., an antibiotic resistance or auxotrophic growth gene) encodes a product that confers growth or survival abilities in selective medium (e.g., containing an antibiotic or lacking an essential nutrient). A negative selectable marker, in contrast, prevents polynucleotide-harboring cells from growing in negative selection medium, when compared to cells not harboring the polynucleotide. A selectable marker may confer both positive and negative selectability, depending upon the medium used to grow the cell. The use of selectable markers in prokaryotic and eukaryotic cells is well known by those of skill in the art.

The term “selector codon” refers to a codon recognized by a modified tRNA in the translation process and not recognized by an endogenous tRNA. The modified tRNA anticodon loop recognizes the selector codon on the mRNA and incorporates its amino acid (either a natural amino acid not normally encoded by a given codon or an unnatural amino acid) at this site in the polypeptide.

The terms “site-specific recombinase” and “sequence-specific recombinase” refer to enzymes that recognize and bind to a short nucleic acid site or sequence and catalyze the recombination of nucleic acid in relation to these sites.

The term “site-specific recombination sequence” refers to a short nucleic acid site or sequence which is recognized by a sequence- or site-specific recombinase and which become the crossover regions during the site-specific recombination event. Examples of sequence-specific recombinase target sites include, but are not limited to, lox sites, frt sites, att sites and dif sites.

As used herein, the term “transfection” means the introduction of a nucleic acid, e.g., an expression vector, into a recipient cell, and is intended to include commonly used terms such as “infect” with respect to a virus or viral vector. The term “transduction” is generally used herein when the transfection with a nucleic acid is by viral delivery of the nucleic acid. The term “transformation” refers to any method for introducing foreign molecules, such as DNA, into a cell. Lipofection, DEAE-dextran-mediated transfection, microinjection, protoplast fusion, calcium phosphate precipitation, retroviral delivery, electroporation, sonoporation, laser irradiation, magnetofection, natural transformation, and biolistic transformation are just a few of the methods known to those skilled in the art which may be used (reviewed, for example, in Mehier-Humbert and Guy, Advanced Drug Delivery Reviews 57: 733-753 (2005)).

The term “unnatural amino acid” refers to any amino acid, modified amino acid, and/or amino acid analogue that is not one of the 20 naturally occurring amino acids or seleno cysteine.

2. Hierarchical Assembly Methods

Synthesis of large, even genome sized nucleic acids, may be produced by a variety of methods available to one of skill in the art based on the disclosure herein. For example, in one embodiment, a modified, partially synthetic, or fully synthetic genome may be produced by substituting into the organism's genome a plurality of polynucleotide constructs (including synthetic polynucleotide constructs and/or modified genomic polynucleotide segments) homologous with the native sequence but implementing the substituted sequences. In another embodiment, an entire genome may be synthesized and then used to replace the naturally occurring genome in the cell. For example, a genome may be synthesized in a bacterial cell and then transferred to another cell, e.g., another bacterial cell, yeast, plant, or eukaryotic cell (including a mammalian cell), by means of conjugation.

In one aspect, the invention provides a method for hierarchical assembly of very large, including genome sized, nucleic acid products. The goal of hierarchical DNA assembly is to reduce the number of steps required to construct large nucleic acids (e.g. chromosomes) from N steps to as little as log₂(N). This could be accomplished by a series of DNA preps and electroporations, but as the DNA pieces get longer than about 100,000 bp (e.g., 100 kbp), DNA fragility becomes a factor. Bacterial conjugation allows transfer of up to 5 Mbp from one bacterium to another bacterium (Li M Z and Elledge S J. Nat Genet. (2005) 37(3): 311-9), or from a bacterium to yeast, plant or mammalian cells (Waters V L Nat Genet. (2001) 29(4): 375-6).

For purposes of comparison, a serial/sequential assembly approach for producing a large nucleic acid product would involve repeated replacements of portions of the genome or plasmid sequence one at a time. For example, replacement of all sequences of an organism's genome may be carried out by serially substituting into the genome polynucleotide constructs having alternating selectable markers. For example, the entire genome of E. coli may be synthesized by introducing 48 polynucleotide constructs of approximately 100 kb into a cell having, for example, λ-red recombinase. Each 100 kb polynucleotide construct contains, for example, either a kanamycin resistant gene (Kan^{R}) or a tetracycline resistant gene (tet^{R}) toward one end of the 100 kb fragment. A 100 kb polynucleotide construct that contains, for example, a Kan^{R} is first introduced into the genome and the cells are selected for kanamycin resistance. In a next step, a second 100 kb polynucleotide construct that contains a tet^{R} gene is introduced into the cell. The tet^{R} 100 kb polynucleotide construct is targeted so that one end destroys the Kan^{R} gene of the first polynucleotide construct and the other end introduces a tet^{R} gene. Cells that are tetracycline resistant and kanamycin sensitive will have the first and second polynucleotide constructs properly incorporated into the genome. Subsequent 100 kb polynucleotide constructs containing alternating Kan^{R} and tet^{R} markers may be introduced into the genome to sequentially replace segments of the genome. In this embodiment, for example, 48 polynucleotide constructs of ˜100 kb may be sequentially introduced with alternating selectable markers to synthesize the entire E. coli genome.

The serial approach requires a separate and sequential step for introduction of each subsequent polynucleotide construct and therefore is very time consuming. For example, the serial approach may be further illustrated with respect to creating a modified, partially synthetic, or fully synthetic E. coli genome. Replacement of the entire genome requires about 48 polynucleotide constructs (e.g., each polynucleotide construct being about 100 kb long for a 4.8 Mbp genome like E. coli) with alternating markers 2 and 3. Recombining these polynucleotide constructs serially into the genome would take 48 stages at about 2 days per stage (e.g., one strain having increasing portions of its genome replaced).

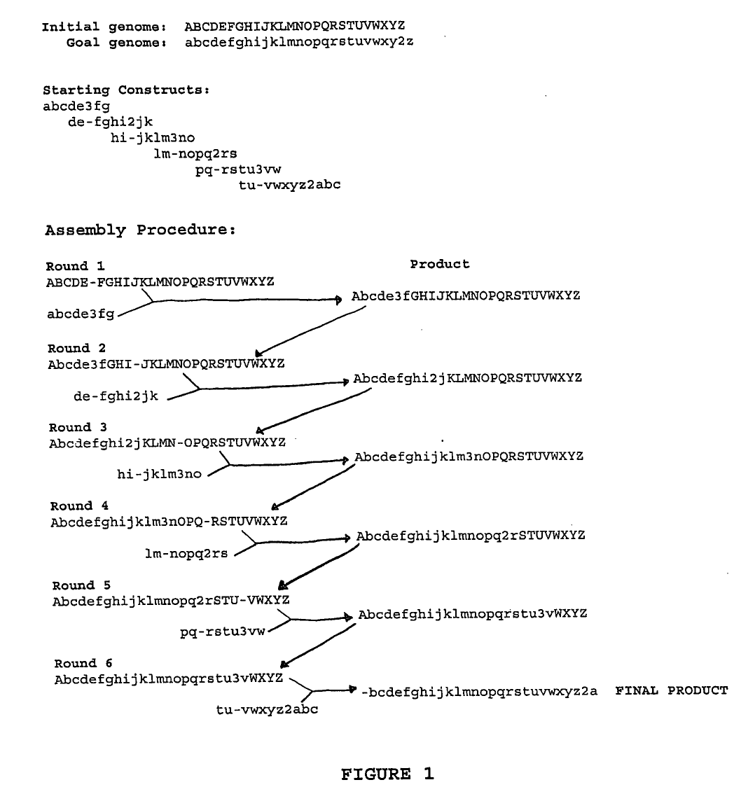

This process is illustrated in FIG. 1 using 6 polynucleotide constructs and hence 6 stages. As shown in FIG. 1, the dashes “-” are simply alignment symbols and do not represent any base pairs. The starting genome (or plasmid polynucleotide) sequence is shown in capital letters and the goal genome (or plasmid polynucleotide sequence) is shown in lower cases letters. It should be noted that since the nucleic acids (e.g., genomes or plasmids) are circular, the goal genome could also be represented, for example, as bcdefghijklmnopqrstuvwxy2za. In this example, selectable genes are referred to by numbers 2 and 3, for example, 2 may be chloramphenical resistance and 3 may be kanamycin resistance. In each case, one of the markers may be selected for to obtain the desired product. The six starting polynucleotide constructs are illustrated below the initial and goal genomes and contain alternating selectable markers (e.g., 2 and 3). As shown in FIG. 1, each polynucleotide construct is sequentially introduced into the genome until the final product is obtained. For example, in Round 1, the first starting polynucleotide construct is homologously recombined into the initial genome and products containing marker 3 are selected. The product formed contains a portion of the first polynucleotide construct which has been incorporated into, and replaced, a segment of the starting genome. The product of round 1 is then combined with the second starting polynucleotide construct and products containing marker 2 are selected. The product formed contains a portion of the first and second polynucleotide constructs which have been incorporated into, and replaced, segments of the starting genome. This process is repeated for six rounds until all six starting polynucleotide constructs have been incorporated into the starting genome to form a genome that has been entirely replaced with the modified and/or synthetic starting polynucleotide constructs thereby forming a modified, partially synthetic or fully synthetic genome (e.g., the final product).

A hierarchical assembly approach may reduce this assembly procedure to only seven stages. Each stage involves multiple strains having one or more segments of their genome replaced with a modified or synthetic polynucleotide construct. For example, at the first stage, 48 different strains each containing a single modified or synthetic polynucleotide construct replacing a segment of the genome are produced. These 48 strains are then combined pairwise to produce 24 strains each comprising two segments of their genome replaced by modified or synthetic polynucleotide constructs. This process is repeated iteratively until all of the desired genome modifications have been made or the entire genome has been substituted, e.g., the 24 strains are combined pairwise to produce 12 strains having four modified or synthetic constructs each, the 12 strains are combined pairwise to produce 6 strains having 8 modified or synthetic constructs each, and so on, thereby producing, 3, 2 and finally 1 strain having all of the desired modifications, or the entire genome replaced, with modified or synthetic polynucleotide constructs. The assembly may be facilitated by bacterial conjugation which permits transfer of large nucleic acids from one cell to another. Alternatively, in certain embodiments, the polynucleotide constructs may be introduced into the cells by a transfection procedure (e.g., electroporation, etc.).

Conjugative transfer of DNA from a bacterium requires trans-acting proteins (e.g. tra genes) and a cis-acting nicking site (origin of transfer or oriT). After nicking the donor genome (or plasmid), a strand-displacing polymerase pushes a DNA copy (starting at the nick primer) into the recipient cell. A variety of oriT sites may be used in conjunction with the methods described herein. For example, ColE1 oriT can be as small as 22 bp (Heeb S, et al. Mol Plant Microbe Interact. (2000) 13(2): 232-7) while oriT sites for the broad host range plasmid RK2 is about 250 bp (Guiney D G, et al. Plasmid. (1988) 20(3): 259-65). Other compatible oriT sites that may be used in accordance with the methods described herein include IncPalpha, F, and R64 (IncI). Some conjugative systems will not mate with the same mating type efficiently. Accordingly, in certain embodiments, the donor sequences may include tra genes (e.g., traF, traG and traJ) which are transferred into the recipient cell that does not contain any tra genes. In an exemplary embodiment, the tra genes may be provided under the control of an inducible or repressible promoter. Additionally, if the oriT is destroyed in the process of transfer, a new oriT site may be made available in the recipient cell for the next round of assembly.

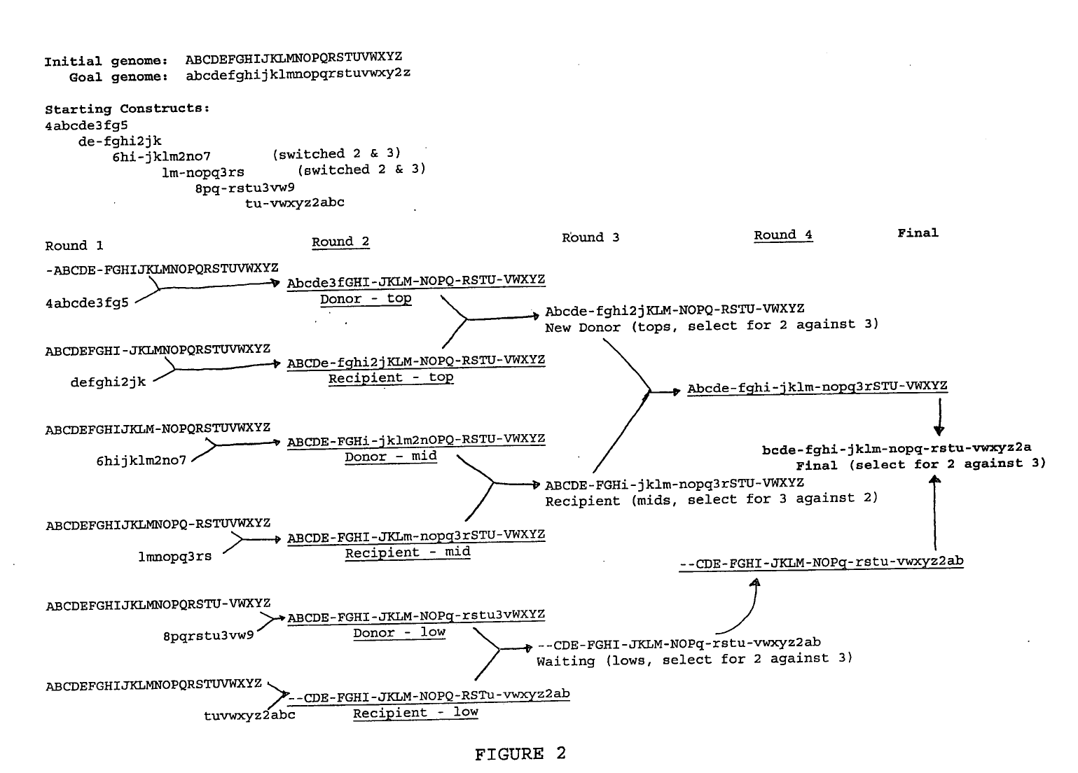

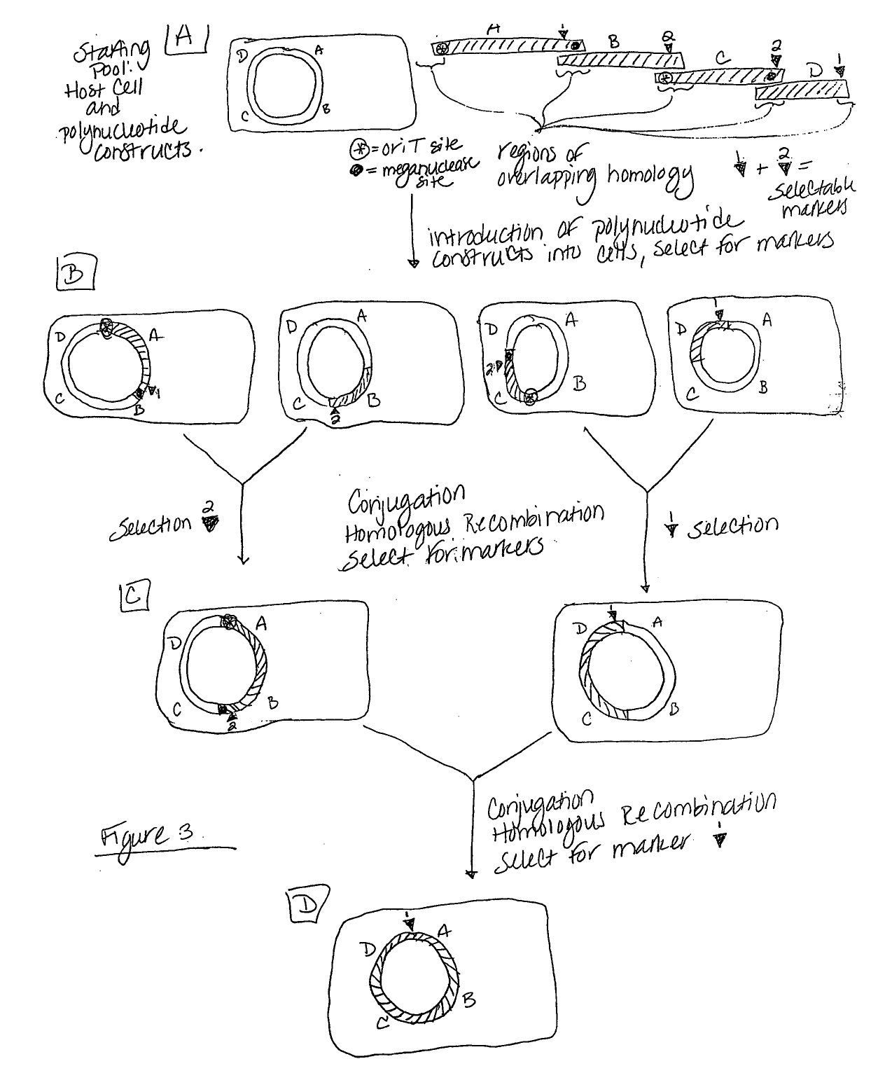

The hierarchical assembly process may be illustrated with reference to FIGS. 2-4. For illustration purposes, the process is represented in FIG. 2 using 6 polynucleotide constructs that require only 4 rounds rather than 6 rounds as was required for the serial assembly approach. Each construct represents a polynucleotide, such as, for example, an approximately 100 kb polynucleotide construct. The markers (e.g., 2 and 3) are arranged in a different order and do not alternate with each sequential segment. Additionally, as shown in FIG. 2, some of the polynucleotide constructs may comprise oriT sites (illustrated as 4, 6, and 8) and meganuclease sites (illustrated as 5, 7, and 9). Any type of meganuclease may be used in association with the methods described herein, including, for example, I-SceI, I-DmoI, I-CreI, and E-DreI (Chevalier B S, et al. Mol Cell. (2002) 10(4): 895-905; Posfai G, et al. Nucleic Acids Res. (1999) 27(22): 4409-15). In an exemplary embodiment, the meganuclease used does not create a double stranded break in the genome of the host cell being used for assembly. The oriT nicking and meganuclease sites direct the recombination machinery by creating recombinogenic ends. At each stage, one of the selectable markers may be used to obtain the desired product. As shown in FIG. 2, the initial genome (upper case) and goal genome (lower case) are illustrated at the top. The six starting polynucleotide constructs are shown below the goal genome. As discussed above, the order of the selectable markers for polynucleotide constructs 3 and 4 have been switched in comparison to the example discussed above for the sequential assembly method. In this embodiment, all of the six starting polynucleotide constructs are first introduced into six separate cells to form products wherein the six polynucleotide constructs are incorporated into, and replace, a segment of the starting genome (Round 1 in FIG. 2). The desired products may be selected using the appropriate marker 2 or 3 for each polynucleotide construct. The cells containing the starting polynucleotide constructs are then combined pairwise to build up larger polynucleotide constructs (Rounds 2-4 in FIG. 2). As shown FIG. 2, Round 2 involves 3 pairwise combinations (indicated as top, mid and low) between donor and recipient strains in each combination. For each pairwise combination, the donor and recipient strains are mixed and the donor cells transfer their DNA to the recipient cells by conjugation. A recipient cell containing the selectable marker from the donor cell is then selected for to produce a new product having two starting polynucleotide constructs incorporated into, and replacing, the genome (Round 2 in FIG. 2). This process is then repeated until the final product is obtained.

This process of hierarchical assembly significantly reduces the time and effort required to produce the final product as compared to the serial/sequential assembly approach. For example, the sequential assembly approach would require approximately 48 stages each taking approximately 2 days, or a total time of about 96 days for producing a synthetic E. coli genome. In contrast, the hierarchical assembly methods provided herein would only require about 7 stages of about 2 days each, or approximately 14 days total. Additionally, cellular conjugation as a means of introducing nucleic acids into a cell is faster than electroporations. Therefore, if selectable markers are chosen that permit both positive and negative selections, then the whole process could be done in liquid cultures (e.g. thyA, galK, and/or supF) and the 7 stages of assembly could be completed in as little as 2 days total time. In exemplary embodiments, such hierarchical assembly methods could be carried out in highly-parallel multi-well plates allowing construction of many such large nucleic acid products, such as genomes, simultaneously.

Each of the genome replacements must produce a viable intermediate. In certain embodiments, each of the starting constructs may be simultaneously introduced into cells and pre-tested for viable intermediates. If any of the starting constructs produces an inviable intermediate, one or more of the constructs may be altered until a complete set of viable intermediates is generated. For example, the polynucleotide constructs may be lengthened or shortened such that a smaller or larger region of the genome is replaced, the location of insertion of the polynucleotide constructs may be shifted upstream or downstream such that a different region of the genome is replaced, and/or the sequence of the polynucleotide construct may be codon remapped, etc.

The hierarchical assembly methods disclosed herein are further graphically illustrated in FIG. 3. FIG. 3A shows the starting materials including a cell with an initial genome (empty) and variety of polynucleotide constructs that together comprise the sequences of the modified genome (hatched; labeled A, B, C, and D). The polynucleotide constructs comprise selectable markers (labeled 1 and 2) and some of them comprise oriT sites (*) and meganuclease cleavage sites (•). Additionally, each polynucleotide construct comprises overlapping regions of sequence homology at each termini (e.g., both termini of each polynucleotide construct have overlapping homology with one other polynucleotide construct). Each polynucleotide construct is separately introduced into a cell, for example, constructs then integrate into the host cell genome by homologous recombination. In certain embodiments, it may be desirable to utilize a host cell that is overexpressing a recombinase and/or comprises a recombinase under the control of an inducible/repressible promoter. Exemplary recombinase systems include, for example, RedE and RecT proteins (from E. coli) or Redα, Redβ and Gam proteins (from lambda). Examples of inducible promoters include, for example, promoters under lac control or rhamnose control (rhaB promoter). The products produced by the introduction of the polynucleotide constructs are a plurality of cells comprising a single integrated polynucleotide construct replacing the wild-type sequence at that location (see FIG. 3B).

Assembly into larger polynucleotide constructs, or whole genome replacement, is achieved by repeated rounds of conjugation and integration (see FIGS. 3C and D). For example, cells comprising polynucleotide constructs with homologous overlapping termini are mixed together and the DNA from one cell (the donor) is transferred into another cell (the recipient cell). The polynucleotide construct is then integrated into the appropriate location in the genome by homologous recombination (e.g., as aligned by the overlapping homologous regions on the polynucleotide constructs). The desired product may be selected using the appropriate selectable marker (see FIG. 3C). This process of conjugation and recombination is repeated until the desired nucleic acid product (or modified, partially synthetic or fully synthetic genome) has been constructed. In alternative embodiments, additional polynucleotide constructs may be introduced into the cells by transfection techniques, such as electroporation, rather than conjuation. In yet other embodiments, various combinations of electroporation and conjugation may be used. The final product may be transferred into a desired cell by conjugation.

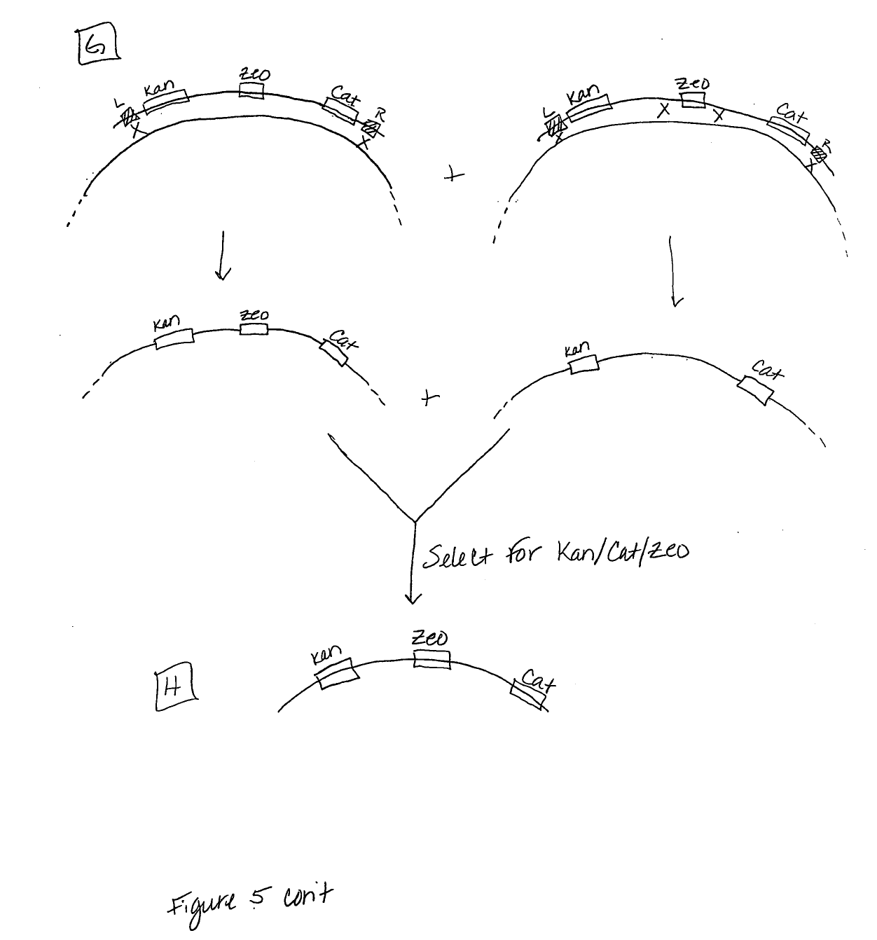

In an exemplary embodiment, segments may be excised from the genome using the methods described in FIG. 5 and then reintroduced into another cell to assemble the desired nucleic acid product. For example, a segment of the genome comprising the integrated polynucleotide construct B (see e.g., FIG. 3B) may be excised from the genome using the methods illustrated in FIG. 5. The excised segment may then be introduced (e.g., by electroporation, etc.) into a cell comprising the integrated polynucleotide construct A to form the intermediate nucleic acid product comprising integrated segments A and B (see e.g., FIG. 3C). This excision and reintroduction process may be repeated until the desired product nucleic acid is formed. In certain embodiments, combinations of excision and conjugation may be used during the assembly process. For example, excision and reintroduction may be used for transferring nucleic acids that are less than 1 Mbp, 500 kbp, or 100 kbp or smaller and conjugation may be used for pieces that are at least 100 kbp, 500 kbp or 1 Mbp or larger. The segments excised from the genome using the methods described in FIG. 5 may comprise the integrated polynucleotide construct and optionally sequences flanking one or both ends of the integrated polynucleotide construct. In certain embodiments, the excised polynucleotide segments may be modified before reintroducing them into a cell.

It should be noted that construction of a genome is provided in the figures merely for purposes of illustration, however, other large polynucleotides may also be constructed in accordance with the methods described herein. The methods utilize a scaffold nucleic acid into which polynucleotide constructs are introduced to replace segments of the scaffold nucleic acid (e.g., by homologous recombination, site specific recombination, etc.). In various embodiments, the scaffold nucleic acid may be a genome or an extrachromosomal nucleotide construct such as a plasmid or artificial chromosome. It should also be understood that the polynucleotide constructs may be designed so as to replace substantially all of the sequences of the scaffold nucleic acid, or only portions of the scaffold nucleic acid. For example, when making only selected sequence changes in particular regions of the scaffold nucleic acid sequence, the methods may only require replacement of select segments of the scaffold nucleic acid while other segments do not require replacement. In other embodiments, the desired sequence changes are scattered throughout the scaffold nucleic acid and replacement of all, or substantially all, of the scaffold nucleic acid may be required.

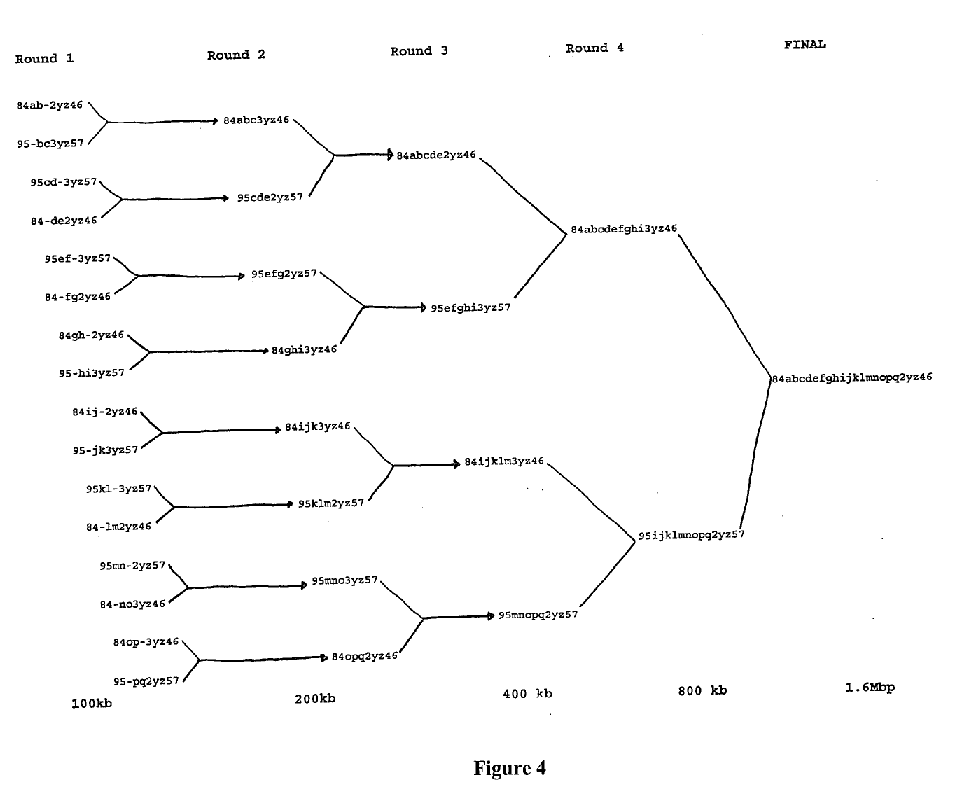

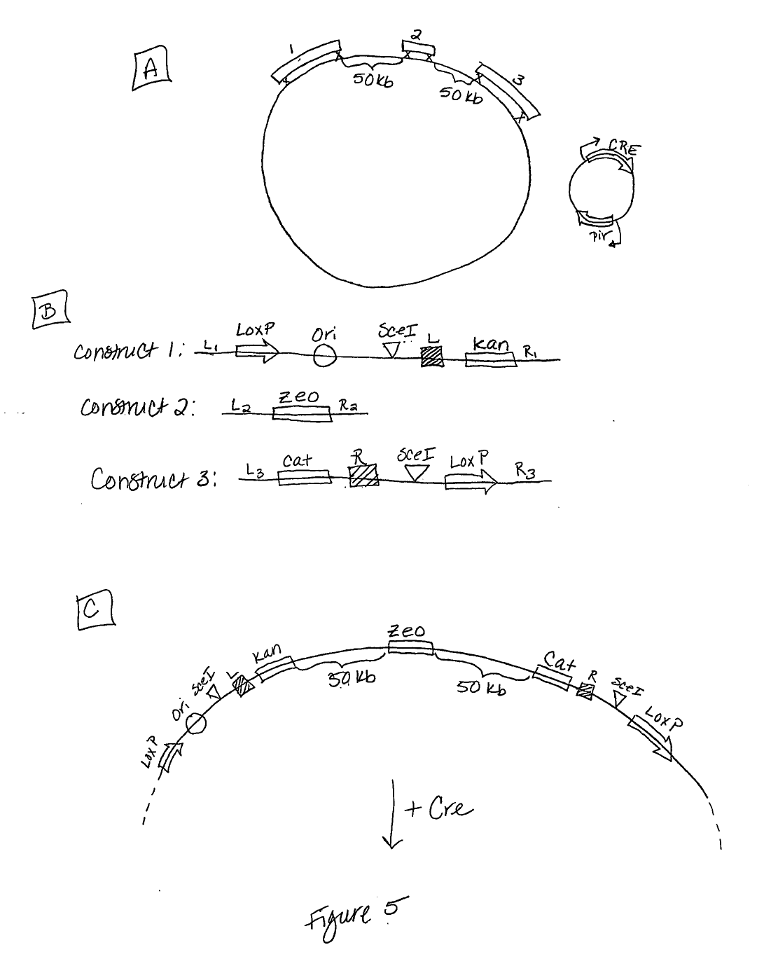

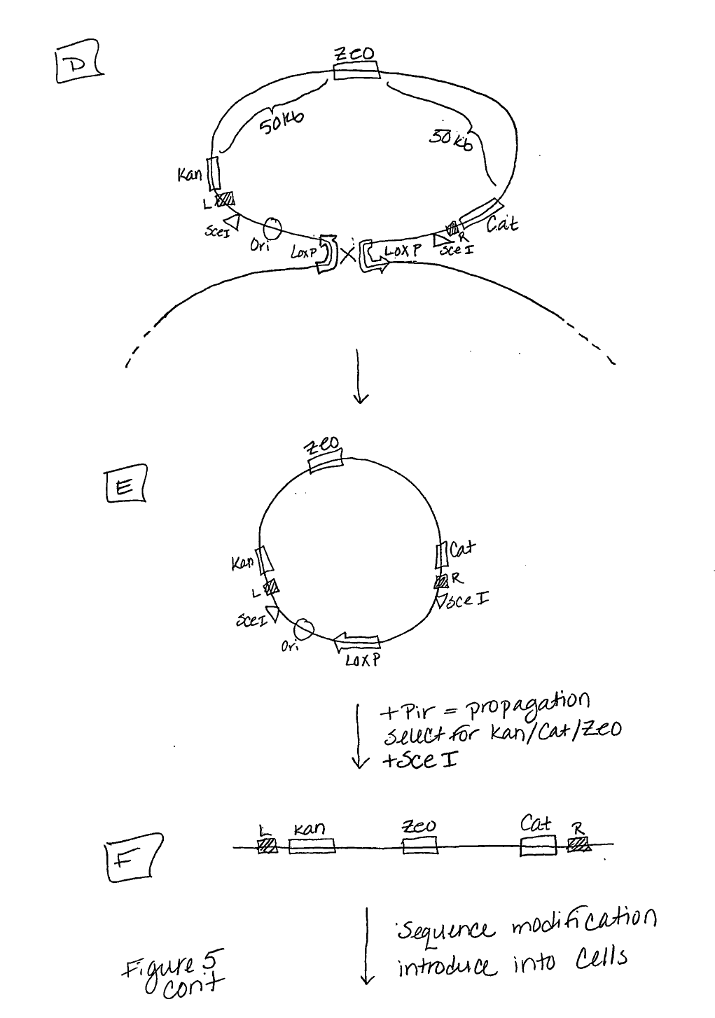

FIG. 4 illustrates another embodiment of a hierarchical assembly method. This embodiment involves two sets of starting polynucleotide constructs each having four types of components including selectable genes (illustrated as 2 and 3 in FIG. 4), meganuclease sites (illustrated as 4 and 5 in FIG. 4), conjugative transfer sites (oriT sites; illustrated as 6 and 7 in FIG. 4), and origins of replication (illustrated as 8 and 9 in FIG. 4). In an exemplary embodiment, these components are conditional components that are function only in desired cells. The 16 starting polynucleotide constructs (illustrated on the left in FIG. 4 under the heading Round 1) are introduced into cells. The cells are then mixed pairwise for a total of 8 pairwise combinations as illustrated in Round 1, FIG. 4. In each case, the cell containing the lower polynucleotide construct of the pair (donor) is transferred into the cell containing the upper polynucleotide construct of the pair (recipient). The cell containing the upper polynucleotide construct contains a meganuclease that recognizes the meganuclease cleavage sites of the incoming (lower) polynucleotide construct. Cleavage by the meganuclease stimulates homologous recombination between the upper and lower polynucleotide constructs. The marker from the lower (incoming) polynucleotide construct is then selected for in each pairwise combination.

Additionally, the upper cell supports replication of the upper polynucleotide construct but not does not support replication of the lower (incoming) polynucleotide construct. This may be achieved using different conditional origins of replication such as, for example, IncX R6K oriγ (dependent on pir protein) or IncPα ori V (dependent on the trfA protein) for the polynucleotide constructs contained in the donor and recipient cells (see e.g., Li M Z and Elledge S J, Nat Genet. 37: 311-9 (2005); Matsumoto-Mashimo C, et al., Res Microbiol. 155: 455-61 (2004)). The recipient strain (e.g., containing the upper polynucleotide construct) expresses a protein that supports replication of the recipient polynucleotide construct (upper construct) but does not express a protein supporting replication of the incoming polynucleotide construct (lower construct). Because the recipient cell only supports replication of the polynucleotide construct originally contained in the cell (or a recombinant product between the recipient and donor polynucleotide constructs), the other portions of the donor polynucleotide construct will be lost (e.g., the portions that do not homologously recombine and incorporate into the recipient polynucleotide construct will be lost). In certain embodiments, negative or counter selectable markers could be placed on the polynucleotide constructs, or the donor genome, to facilitate loss of the unwated portions of the donor polynucleotide construct that are transferred to the recipient strain. The negative selectable marker may be incorporated into the polynucleotide construct outside of the region that homologously recombines with the receipient genome such that portions of the polynucleotide construct not incorporated into the genome may be removed by negative selective pressure and/or cells which have incorporated undesired regions of the polynucleotide construct into the recipient genome may be removed using negative selective pressure. Similarly, when using conjugation based assembly methods, donor cells having a polynucleotide incorporated into the genome, may also have a negative selectable marker incorporated into the donor genome outside of the polynucleotide construct. In exemplary embodiments, the negative selectable marker may be incorporated into the donor genome downstream from the polynucleotide construct. This permits removal of portions of the donor genome introduced into the recipient cell but not incorporated into the recipient genome to be removed by negative selective pressure and/or cells which have incorporated undesired regions of the donor genome into the recipient genome may be removed using negative selective pressure.

The results from the pairwise combinations in Round 1 are shown under the heading Round 2. The recipient cells from the first round now contain polynucleotide constructs that have flanking markers from the recipient polynucleotide construct (upper) and a selectable marker from the donor polynucleotide construct (lower). This process is repeated using further rounds of pairwise mixing, conditional cleavage of the incoming polynucleotide construct at the meganuclease site, selection of the incoming selectable marker, and facilitated loss of the unwanted portions of the incoming polynucleotide construct using a conditional origin of replication and/or a negative selectable marker (see e.g., Rounds 2-4 in FIG. 4) until the desired product is achieved.

It should be understood that for purposes of illustration only, FIG. 4 utilized a combination of two selectable markers, two meganuclease cleavage sites, two conjugative transfer elements and two origins of replication. In certain embodiments, it may be desirable to use 1, 2, 3, 4, 5, 6, 7, 8, 9, 10, or more, different selectable markers, meganuclease cleavage sites, conjugative transfer elements, and/or origins of replication.

Furthermore, for purposes of illustration only, FIG. 4 utilized double crossover homologous recombination mediated by meganuclease cleavage and lambda-red in E. coli. However, the hierarchical assembly methods described herein may be carried out using a variety of recombination systems including ‘single crossover integration’ (involving either specific sequences like phage integrases or general homologous recombination). The recombination events can be clean as illustrated in FIG. 4 or can leave a small scar (insertion and/or deletion of one or more nucleotides). Similar strategies are applicable to other microbial, viral, plant or animal DNA assembly as described further herein.

In various embodiments, the hierarchical assembly methods described in FIGS. 2 and 4 may be carried out using bacterial conjugation, excision (see e.g., FIG. 5) followed by reintroduction, or combinations thereof to transfer polynucleotide constructs between cells.

In certain embodiments, various combinations of selectable, counterselectable and non-marker sequences may be used in association with the assembly methods described herein. In one embodiment, polynucleotide constructs containing one or more selectable markers may be used and desired products may be selected by assaying cells for the presence of the selectable marker. For example, a first polynucleotide construct comprising selectable marker X may be introduced into a cell. The presence of selectable marker X ensures that the cell contains the first polynucleotide construct. For example, if X is a kanamycin resistance gene (kan^{R}), the presence of the first polynucleotide construct may be selected for by growing the cells in the presence of kanamycin. Cells that survive in the presence of kanamycin comprise the desired first polynucleotide construct. A second polynucleotide construct comprising selectable marker Y may then be introduced into the cell and determining the presence of selectable marker Y ensures that the cells now contain the second polynucleotide construct.

In another embodiment, polynucleotide constructs may comprise both selectable and counterselectable markers. For example, a first polynucleotide construct comprising sequence X having both a selectable marker and a counterselectable marker may be introduced into the cell. The presence of the first polynucleotide construct in the cell may be determined by assaying for the presence of the selectable marker and/or the presence of the counterselectable marker. For example, the presence of the selectable marker may be determined as described above for kan^{R}. Presence of the counterselectable marker may be determined, for example, by detecting cells that are viable in the absence of the counterselectable conditions and inviable in the presence of the counterselectable conditions. For example, if the counterselectable marker is the HSV-tk gene, cells expressing the HSV-tk gene and grown in the presence of gancyclovir will be killed or inviable. A second polynucleotide construct comprising sequence Y may then be introduced into the cells. Proper joining of the second polynucleotide construct with the first polynucleotide construct, for example, by homologous recombination, site specific recombination, etc., leads to destruction of at least a portion of sequence X. For example, proper joining of the second polynucleotide construct with the first polynucleotide construct may lead to destruction of the counterselectable marker of sequence X. In this case, the presence of the desired product may be determined by assaying for the absence of the counterselectable marker of sequence X. In various embodiments, sequence Y may be a non marker sequence, a selectable marker, or may comprise both a selectable and a counterselectable marker. When Y is a non-marker sequence, sequence Y represent a portion of the second polynucleotide construct to be joined to the first polynucleotide but does not contain any sequence specifically to be used for selection purposes. If sequence Y comprises a selectable marker, proper joining of the first and second polynucleotide constructs may be determined by selecting for the absence of the counterselectable marker X and the presence of selectable marker Y. If sequence Y comprises a selectable marker and a counterselectable marker, proper joining of the first and second polynucleotide constructs may determined by selecting for the absence of the counterselectable marker of X and the presence of the selectable and/or counterselectable marker of Y. In such embodiments, the selectable and counterselectable markers of sequences X and Y may be different so that different selection or counterselection may be used to selectively detect the presence of the different polynucleotide constructs. Various combinations and/or repetitions of the foregoing selection strategies may be used in association with the assembly methods described herein.

In yet another embodiment, the invention provides methods for assembly of a polynucleotide product comprising selecting an initiating polynucleotide construct and contacting the initiating polynucleotide construct with a next polynucleotide construct in the presence of a recombination system, wherein said next polynucleotide construct is double stranded and a terminal region of said next polynucleotide construct comprises substantial sequence homology with a terminal region of said initiating polynucleotide construct, and wherein said next polynucleotide construct is joined to said initiating polynucleotide construct by homologous recombination at the terminal regions having substantial sequence homology. This process may be reiteratively repeated with additional next polynucleotide constructs to extend the initiating polynucleotide construct by successive rounds of homologous recombination until a desired product polynucleotide is formed. The homologous recombination may be carried out in vitro or in vivo, e.g., in a cell naturally comprising or engineered to comprise a recombination system. The assembly methods may be carried out in a serial manner or hierarchical manner as described more fully above. When conducting hierarchical assembly it may be desirable to utilize an in vivo recombination system and optionally conjugative transfer to facilitate manipulation and introduction of polynucleotide constructs. In various embodiments, an assembly strategy involving successive rounds of homologous recombination may utilize polynucleotide constructs comprising one or more selectable and/or counterselectable markers as described more fully herein. In certain embodiments, the assembly strategy involving successive rounds of homologous recombination may utilize a nucleic acid scaffold to facilitate assembly, such as, for example, a genome, an extrachromosomal nucleic acid, or any other type of nucleic acid scaffold useful for in vivo or in vitro assembly methods. When using a nucleic acid scaffold for assembly of a polynucleotide product, the polynucleotide product may optionally be excised or removed from the scaffold following assembly as appropriate for the product that has been synthesized. Polynucleotide constructs useful for successive recombination assembly strategies may come from any source as described herein, for example, they may be fully or partially synthetic, they may be excised from a genome, and/or they may be excised from a genome followed by sequence modification, etc.

Exemplary selectable markers that may be used in association with the assembly methods and genome excision methods described herein include, for example, drug resistance (chloramphenicol, kanamycin, ampicillin, tetracycline, bleomycin, hygromycin, neomycin, zeomycin, trimethoprim, gentamicin, spectinomycin, streptomycin etc.), nutritional/auxotrophic (thyA, galK, hisD, etc.), surface properties (omp), chemotaxis (che), fluorescence (green fluorescent protein or gfp), or luminescence (lux). Alternatively, a contiguous region of DNA with one or more markers can encode one or more positive and/or negative selectable markers. For example, a single gene (e.g. supF) can suppress stop, frameshift or missense mutations in any of the above markers and one or more negative markers, including restriction endonuclease genes (EcoRI), toxin genes (cea, kil, ccdB, MazF, RelE), nutrition (thyA, galK), or drug sensitivity (tetAR, rpsL, sacB). Alternatively, a regulatory gene (e.g., ta-RNA) can regulate one or more of positive and/or negative selectable markers. In certain embodiments a negative selectable marker or counterselectable marker may encode an enzymatic activity whose expression is cytotoxic to the cell when grown in an appropriate selective medium. For example, the HSV-tk gene and the dt gene may be used as a negative selectable markers. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme. Similarly, the expression of the dt gene selects against cells capable of expressing the Diphtheria toxin. Other selectable markers are described in the literature (see, for example, Kaufman, Meth. Enzymol., 185:487 (1990); Kaufman, Meth. Enzymol., 185:537 (1990); Srivastava and Schlessinger, Gene, 103:53 (1991); Romanos et al., in DNA Cloning 2: Expression Systems, 2^{nd} Edition, pages 123-167 (IRL Press 1995); Markie, Methods Mol. Biol., 54:359 (1996); Pfeifer et al., Gene, 188:183 (1997); Tucker and Burke, Gene, 199:25 (1997); Hashida-Okado et al., FEBS Letters, 425:117 (1998); and U.S. Pat. No. 5,464,764). Exemplary negative selectable or counterselectable markers include but are not limited to lethal genes, such as bar (barstar), those encoding a restriction enzyme (a gene encoding a corresponding methylase), or those encoding nuclease colicins, e.g., E9 DNAse, and colicin RNases and tRNases, or gyrase A, as well as MazF(ChpAK), Doc (Phd), ParE, PasB, StbOrf2, HigB, z, RelE, Txe, YeoB, SacB, KilA, KorA, KorB, Kid (Kis), PemK (PemI), Hok (Sok), Dcc (Pno), CcdB (CcdA), F′ plasmid, and the like.

Exemplary restriction enzymes that may be used in association with the hierarchical assembly methods and genome excision methods described herein include, but are not limited to, type I enzymes, type II enzymes, type IIS enzymes, type III enzymes and type IV enzymes.

Exemplary meganucleases/meganuclease cleavage sites that may be used in association with the hierarchical assembly methods and genome excision methods described herein include, for example, I-SceI (cut site: TAGGG_ATAAˆCAGGGTAAT), I-DmoI (cut site: GCCTTGCCGG_GTAAˆGTTCCGGCGCG), I-CreI (cut site: CAAAACGTC_GT GAˆGACAGTTTGGT), and I-DreI-3 (cut site: CAAAACGTC_GTAAˆGTTCCGGCG CG) (see e.g., Chevalier B S, et al., Mol Cell. 2002 10: 895-905 (2002)).

Exemplary conditional conjugative transfer elements (or origins of transfer, oriT) that may be used in accordance with the hierarchical assembly methods and genome excision methods described herein include, for example, ColE1 oriT, incBCD oriT, and R64 (IncI). The colE1 oriT can be as small as 22 base pairs (see e.g., Heeb S, et al. Mol Plant Microbe Interact. 13: 232-7 (2000)) and IncPa RP4—RK2 can be as small as 250 base pairs (see e.g., Guiney D G et al., Plasmid 20: 259-65 (1988)).

Conditional origins of replication are origins that require the presence or expression of a trans-acting factor in the host cell for replication. A variety of conditional origins of replication functional in prokaryotic hosts (e.g., E. coli) are known to the art. Exemplary conditional origins of replication that may be used in accordance with the hierarchical assembly methods and genome excision methods described herein include, for example, the R6Kγ origin. The R6Kγ origin requires a trans-acting factor, the π protein supplied by the pir gene (Metcalf et al. (1996) Plasmid 35: 1). E. coli strains containing the pir gene will support replication of R6Kγ origins to medium copy number. A strain containing a mutant allele of pir, pir-116, will allow an even higher copy number of constructs containing the R6Kγ origin (i.e., 15 copies per cell for the wild type versus 250 copies per cell for the mutant). E. coli strains that express the pir or pir-116 gene product include BW18815 (ATCC 47079; this strain contains the pir-116 gene), BW19094 (ATCC 47080; this strain contains the pri gene), BW20978 (this strain contains the pir-116 gene), BW20979 (this strain contains the pir gene), BW21037 (this strain contains the pir-116 gene) and BW21038 (this strain contains the pir gene) (Metcalf et al., supra).

Other conditional origins of replication suitable for use in the hierarchical assembly methods and/or genome excision methods described herein include, but are not limited to: the RK2 oriV from the plasmid RK2 (ATCC 37125). The RK2 oriV requires a trans-acting protein encoded by the trfA gene (Ayres et al. (1993) J. Mol. Biol. 230: 174); the bacteriophage P1 ori which requires the repA protein for replication (Pal et al. (1986) J. Mol. Biol. 192: 275); the origin of replication of the plasmid pSC101 (ATCC 37032) which requires a plasmid encoded protein, repA, for replication (Sugiura et al. (1992) J. Bacteriol. 175: 5993). The pSC101 ori also requires IHF, an E. coli protein. E. coli strains carrying the himA and himD (hip) mutants (the him and hip genes encode subunits of IHF) cannot support pSC101 replication (Stenzel et al. (1987) Cell 49: 709); the bacteriophage lambda ori which requires the lambda O and P proteins (Lambda I I, Hendrix et al. Eds., Cold Spring Harbor Press, Cold Spring Harbor, N.Y. (1983)); pBR322 and other ColE1 derivatives will not replicate in polA mutants of E. coli and therefore, these origins of replication can be used in a conditional manner (Grindley and Kelley (1976) Mol. Gen. Genet. 143: 311); and replication-thermosensitive plasmids such pSU739 or pSU300 which contain a thermosensitive replicon derived from plasmid pSC101, rep pSC101^{ts} which comprises oriV (Mendiola and de la Cruz (1989) Mol. Microbiol. 3: 979 and Francia and Lobo (1996) J. Bact. 178: 894). pSU739 and pSU300 are stably maintained in E. coli strain DH5α (Gibco BRL) at a growth temperature of 30° C. (42° C. is non-permissive for replication of this replicon).

Other conditional-origins of replication, including other temperature sensitive replicons, are known in the art and may be employed in accordance with the methods described herein.

The hierarchical assembly methods described herein may be carried out in vitro or in vivo. In certain embodiments, a scaffolding nucleic acid (e.g., a genome or extraschromosomal nucleic acid such as a plasmid or artifical chromosome) may be used although this is not necessary in every case. When conducting hierarchical assembly in vivo (e.g., using cells), polynucleotide constructs/segments may be introduced into the cells at the various rounds biologically (e.g. conjugation or transduction), chemically (e.g. transfection or transformation), optically or electrically or various combinations thereof within the same or different rounds.

In certain embodiments, the polynucleotide constructs used herein for hierarchical assembly methods may be designed with the aid of a computer to anticipate the complementary markers in all stages of the assembly process. Furthermore, computer aided design of the polynucleotide constructs will facilitate design of the regions of homology located near the meganuclease cut sites that typically will have one end that is homologous to the scaffold being used for construction (e.g., genome or extrachromosomal scaffold) and the other end homologous to a portion of the scaffold that has been modified by previously incorporated polynucleotide constructs. Design of polynucleotide constructs useful for hierarchical assembly may be facilitated by the aid of a computer program such as, for example, DNA Works (Hoover and Lubkowski, Nucleic Acids Res. 30: e43 (2002), Gene2Oligo (Rouillard et al., Nucleic Acids Res. 32: W176-180 (2004) and world wide web at berry.engin.umich.edu/gene2oligo), and Jayaraj et al, Nucleic Acids Research 33: 3011-3016 (2005).

It should be understood that in certain instances the polynucleotide constructs described herein for purposes of genome modification and/or assembly were referred to as ˜100 kb polynucleotide constructs for purposes of illustration only (e.g., FIGS. 1-4). The polynucleotide constructs used to construct a nucleic acid product, such as, for example, a modified, partially synthetic, or fully synthetic genome may be of any size. Typically polynucleotide constructs will be chosen to minimize the number of rounds needed to assemble the desired large product nucleic acid while still permitting the polynucleotide constructs to be easily manipulable without being overly susceptible to shearing. In various embodiments, polynucleotide constructs useful for hierarchical assembly may be at least about 5 kb, 10 kb, 25 kb, 50 kb, 75 kb, 100 kb, 150 kb, 200 kb, 250 kb, 300 kb, 500 kb, 1 Mbp, or larger.

3. Methods for Producing Polynucleotide Constructs for Genome Assembly or Modification

The hierarchical assembly methods described herein require polynucleotide constructs for assembly of large product nucleic acids, such as, for example, a modified, partially synthetic, or fully synthetic genome. Polynucleotide constructs useful for the methods described herein may be obtained from a variety of sources such as, for example, DNA libraries, BAC libraries, de novo chemical synthesis, or excision and modification of a genomic segment. The sequences obtained from such sources may then be modified using standard molecular biology and/or recombinant DNA technology to produce polynucleotide constructs having desired modifications for reintroduction into, or construction of, a large product nucleic acid, including a modified, partially synthetic or fully synthetic genome. Exemplary methods for modification of polynucleotide sequences obtained from a genome or library include, for example, site directed mutageneis; PCR mutagenesis; inserting, deleting or swapping portions of a sequence using restriction enzymes optionally in combination with ligation; homologous recombination in vitro or in vivo; and site-specific recombination; or various combinations thereof.

In other embodiments, the polynucleotide constructs useful in accordance with the methods described herein may be synthetic polynucleotides. Synthetic polynucleotides may be produced using a variety of methods such as high throughput oligonucleotide assembly techniques described in Zhou et al. Nucleic Acids Research, 32: 5409-5417 (2004); Richmond et al. Nucleic Acids Research 32: 5011-5018 (2004); Tian et al. Nature 432: 1050-1054 (2004); Carr et al. Nucleic Acids Research 32: e162 (2004); PCT Publication No. WO 2005/059096; and copending applications having Ser. Nos. 11/068,321, 11/067,812, and 11/254,250. For example, oligonucleotides having complementary, overlapping sequences may be synthesized on a chip and then eluted off. The oligonucleotides then are induced to self assemble based on hybridization of the complementary regions.

In yet another embodiment, polynucleotide constructs useful in accordance with the hierarchical assembly methods described herein may be excised from the genome and then modified as described above. One exemplary method for excising polynucleotide segments from a genome is described in Yoon, Y G et al., Genetic Analysis: Biomolecular Engineering 14: 89-95 (1998). Another exemplary method for excising polynucleotide segments from a genome is illustrated in FIGS. 5A-H. As illustrated in FIG. 5A, a genome is first modified to include the three polynucleotide constructs illustrated in FIG. 5B. The cell containing the genome may additionally contain sequences encoding a site-specific recombinase (e.g., Cre) and/or a replication initiation protein (e.g., pir) under the control of a constitutive or regulatable promoter. The recombinase and/or replication initiation protein may be incorporated into the genome of the cell or may be contained on the same or different extrachromosomal sequences (e.g., plasmids) in the host cell. FIG. 5B illustrates the three polynucleotide constructs that are introduced into the genome. Polynucleotide construct 1 contains a region L₁ that is homologous to a portion of the host genome, a first site-specific recombination site (e.g., LoxP), an origin of replication (Ori), a meganucelase cleavage site (e.g, SceI), a region L that is homologous to the host genome to be used for purposes of reintroducing the genomic fragment into the genome, a first selectable marker (e.g., kanamycin), and a region R₁ that is homologous to a region of the host cell genome. The regions L₁ and R₁ are used for purposes of integrating polynucleotide construct 1 into the genome by homologous recombination at a desired location. Polynucleotide construct 2 contains a second selectable marker (e.g., zeo) flanked by two regions of homology with the host cell genome (e.g., L₂ and R₂) for purposes of integrating the polynucleotide construct into the host cell genome. Polynucleotide construct 3 contains a region L₃, a third selectable marker (e.g., chloramphenicol acetyl transferase or cat), a region R that is homologous to the host genome to be used for purposes of reintroducing the genomic fragment into the genome, a meganuclease cleavage site (may be the same or different from the site used in polynucleotide construct 1), a second site-specific recombination site (e.g., LoxP) in the same orientation as the first site-specific recombination sequence present in polynucleotide construct 1, and a region R₃. The regions L₃ and R₃ are used for purposes of integrating polynucleotide construct 3 into the genome by homologous recombination at a desired location. FIG. 5C illustrates the genome after incorporation of the three polynucleotide constructs shown in FIG. 5B. Polynucleotide constructs 1 and 2 are incorporated at locations flanking the region that is desired to be excised from the genome. For purposes of illustration, polynucleotide constructs 1 and 2 are shown to be incorporated into the genome flanking a 100 kb region. However, the polynucleotide constructs may be incorporated at locations closer or farther apart to excise smaller or larger segments from the genome, such as, for example, segments of about 5 kb, 10 kb, 25 kb, 50 kb, 75 kb, 100 kb, 150 kb, 200 kb, 250 kb, 300 kb, 500 kb, 1 Mbp, or larger.

After incorporating the three polynucleotide constructs into the genome as shown in FIG. 5C, the genome is then exposed to a recombinase such as Cre. For example, the recombinase may be constitutively expressed in the host cell, expression of the recombinase may be induced in the host cell, or the genome may be transferred to a cell expressing the recombinase (e.g., by conjugation). As shown in FIG. 5D, the recombinase stimulates site-specific recombination between the site-specific recombination sites of polynucleotide constructs 1 and 3 thereby forming a circular segment of DNA that has been excised from the genome (see FIG. 5E). The excised circular polynucleotide segment (FIG. 5E) may then be amplified by exposing the circular polynucleotide segment to a replication initiation protein. For example, the replication initiation protein may be constitutively expressed in the host cell, expression of the replication initiation protein may be induced in the host cell, or the genome may be transferred to a cell expressing the replication initiation protein (e.g., by conjugation). Presence of the excised polynucleotide segment may be selected for using one or more of the selectable markers (e.g., illustrated as kan, cat and/or zeo). After amplification of the excised polynucleotide segment, the sequence of the segment may be modified before or after cleavage of the segment with a meganuclease to linearize the segment thereby forming a polynucleotide construct useful for the assembly processes described herein. Sequence modifications may be any type of modification including, insertions, deletions, or point mutations and may be made using standard moleculae biology and/or recombinant DNA techniques. The circular polynucleotide construct may then be linearized by exposing the polynucleotide construct to a meganuclease before or after introduction of the polynucleotide construct into a cell (e.g., may occur in vitro or in vivo). After linearization and introduction of the polynucleotide construct into the cell (in either order), the polynucleotide construct is incorporated into the genome, for example, by homologous recombination using the L and R regions homologous to the genome. To ensure that the polynucleotide construct was integrated in a contiguous manner, cells that express all three markers may be selected for. In the event that multiple cross overs occur, the middle marker (e.g., illustrated as zeo) may be lost and therefore cells incorporating the polynucleotide construct at noncontiguous locations would be lost using a selection for the middle marker. The final product is shown in FIG. 5H in which the polynucleotide segment that was excised from the genome has been modified and reincorporated into, and replaced a segment of, the wild-type genome.

Site-specific recombinases are enzymes that recognize a specific DNA site or sequence (e.g., a site-specific recombination sequence) and catalyze the recombination of DNA in relation to these sites. Site-specific recombinases are employed for the recombination of DNA in both prokaryotes and eukaryotes. Examples of site-specific recombination include, but are not limited to: 1) chromosomal rearrangements that occur in Salmonella typhimurium during phase variation, inversion of the FLP sequence during the replication of the yeast 2 μm circle, and in the rearrangement of immunoglobulin and T cell receptor genes in vertebrates, 2) integration of bacteriophages into the chromosome of prokaryotic host cells to form a lysogen, and 3) transposition of mobile genetic elements (e.g., transposons) in both prokaryotes and eukaryotes. A site-specific recombinase is an enzyme that recognizes short DNA sequences that become the crossover regions during the recombination event and include recombinases, transposases, and integrases. Examples of site-specific recombination sequences include, for example, lox sites, frt sites, att sites and dif sites.

The genome excision and hierarchical assembly methods described herein may utilize, but are not limited to, lox sites (e.g., loxP sites) and the recombination of these sequences using the Cre recombinase of bacteriophage P1. The Cre protein catalyzes recombination of DNA between two loxP sites and is involved in the resolution of P1 dimers generated by replication of circular lysogens (Sternberg et al. (1981) Cold Spring Harbor Symp. Quant. Biol. 45: 297). Cre can function in vitro and in vivo in many organisms including, but not limited to, bacteria, fungi, and mammals (Abremski et al. (1983) Cell 32: 1301; Sauer (1987) Mol. Cell. Biol. 7: 2087; and Orban et al. (1992) Proc. Natl. Acad. Sci. 89: 6861). The loxP sites may be present on the same DNA molecule or they may be present on different DNA molecules; the DNA molecules may be linear or circular or a combination of both. The loxP site consists of a double-stranded 34 bp sequence which comprises two 13 bp inverted repeat sequences separated by an 8 bp spacer region (Hoess et al. (1982) Proc. Natl. Acad. Sci. USA 79: 3398 and U.S. Pat. No. 4,959,317). The internal spacer sequence of the loxP site is asymmetrical and thus, two loxP sites can exhibit directionality relative to one another (Hoess et al. (1984) Proc. Natl. Acad. Sci. USA 81: 1026). When two loxP sites on the same DNA molecule are in a directly repeated orientation, Cre excises the DNA between these two sites leaving a single loxP site on the DNA molecule (Abremski et al. (1983) Cell 32: 1301). If two loxP sites are in opposite orientation on a single DNA molecule, Cre inverts the DNA sequence between these two sites rather than removing the sequence. Two circular DNA molecules each containing a single loxP site will recombine with one another to form a mixture of monomer, dimer, trimer, etc. circles. The concentration of the DNA circles in the reaction can be used to favor the formation of monomer (lower concentration) or multimeric circles (higher concentration).

Circular DNA molecules having a single loxP site will recombine with a linear molecule having a single loxP site to produce a larger linear molecule. Cre interacts with a linear molecule containing two directly repeating loxP sites to produce a circle containing the sequences between the loxP sites and a single loxP site and a linear molecule containing a single loxP site at the site of the deletion.

The Cre protein has been purified to homogeneity (Abremski et al. (1984) J. Mol. Biol. 259: 1509) and the cre gene has been cloned and expressed in a variety of host cells (Abremski et al. (1983), supra). Purified Cre protein is available from a number of suppliers (e.g., Novagen and New England Nuclear/DuPont).

The Cre protein also recognizes a number of variant or mutant lox sites (variant relative to the loxP sequence), including the loxB, loxL and loxR sites which are found in the E. coli chromosome (Hoess et al. (1982), supra). Other variant lox sites include loxP511 (Hoess et al. (1986), Nucleic Acids Res. 14: 2287-300), loxC2 (U.S. Pat. No. 4,959,317), loxΔ86, loxΔ117, loxP2, loxP3, loxP23, loxS, and loxH. Cre catalyzes the cleavage of the lox site within the spacer region and creates a six base-pair staggered cut (Hoess and Abremski (1985) J. Mol. Biol. 181: 351). The two 13 bp inverted repeat domains of the lox site represent binding sites for the Cre protein. If two lox sites differ in their spacer regions in such a manner that the overhanging ends of the cleaved DNA cannot reanneal with one another, Cre cannot efficiently catalyze a recombination event using the two different lox sites. For example, it has been reported that Cre cannot recombine (at least not efficiently) a loxP site and a loxP511 site; these two lox sites differ in the spacer region. Two lox sites which differ due to variations in the binding sites (i.e., the 13 bp inverted repeats) may be recombined by Cre provided that Cre can bind to each of the variant binding sites. The efficiency of the reaction between two different lox sites (varying in the binding sites) may be less efficient than that between two lox sites having the same sequence (the efficiency, will depend on the degree and the location of the variations in the binding sites). For example, the loxC2 site can be efficiently recombined with the loxP site, as these two lox sites differ by a single nucleotide in the left binding site.

Other exemplary site-specific recombinases that may be employed in accordance with the genome excision and/or hierarchical assembly methods described herein include, for example: the FLP recombinase of the 2μ plasmid of Saccharomyces cerevisiae (Cox (1983) Proc. Natl. Acad. Sci. USA 80: 4223) which recognizes the frt site. Like the loxP site, the frt site comprises two 13 bp inverted repeats separated by an 8 bp spacer. The FLP gene has been cloned and expressed in E. coli (Cox, supra) and in mammalian cells (PCT Publication No.: WO 92/15694) and has been purified (Meyer-Lean et al. (1987) Nucleic Acids Res. 15: 6469; Babineau et al. (1985) J. Biol. Chem. 260: 12313; and Gronostajski and Sadowski (1985) J. Biol. Chem. 260: 12328); the Int recombinase of bacteriophage lambda (with or without Xis) which recognizes att sites (Weisberg, et al., “Site-specific recombination in Phage Lambda,” In: Lambda I I, Hendrix, et al. Eds., Cold Spring Harbor Press, Cold Spring Harbor, N.Y. (1983) pp. 211-250); the xerC and xerD recombinases of E. coli which together form a recombinase that recognizes the 28 bp dif site (Leslie and Sherratt (1995) EMBO J. 14: 1561); the Int protein from the conjugative transposon Tn916 (Lu and Churchward (1994) EMBO J. 13: 1541); TpnI and the β-lactamase transposons (Levesque (1990) J. Bacteriol. 172: 3745); the Tn3 resolvase (Flanagan et al. (1989) J. Mol. Biol. 206: 295 and Stark et al. (1989) Cell 58: 779); the SpoIVC recombinase of Bacillus subtilis (Sato et al. (1990) J. Bacteriol. 172: 1092); the Hin recombinase (Galsgow et al. (1989) J. Biol. Chem. 264: 10072); the Cin recombinase (Hafter et al. (1988) EMBO J. 7: 3991); and the immunoglobulin recombinases (Malynn et al. Cell (1988) 54: 453).

4. Exemplary Applications

The hierarchical assembly methods described herein may be used, for example, to construct large polynucleotide constructs that may not be constructed ex vivo (or at least not easily constructed ex vivo) due to shearing and other difficulties in manipulating large nucleotide constructs. The methods may also be used for making genome wide nucleotide alterations in a cell. For example, producing a genome having a plurality of sequence alterations scattered throughout the genome may be achieved using the hierarchical assembly methods described herein. Such methods may be used to produce a genome having a plurality of specific and predetermined nucleotide or codon substitutions, for example, at least about 50, 100, 200, 500, 750, 1000, 2000, 5000, 10000, or more, specific nucleotide or codon alterations at different and predetermined locations throughout the genome, e.g., the changes are spread across at least 25%, 30%, 45%, 50%, 60%, 70%, 75%, 80%, 85%, 90%, 95%, 99%, or more of the genome. To determine the percentage of the genome comprising the nucleotide or codon alterations, the smallest number of nucleotides encompassing all of the alterations and any sequences between the alterations may be divided by the total number of nucleotides in the genome. In an exemplary embodiment, a substantial proportion of the nucleotide or codon alterations are non-contiguous, e.g., at least about 25%, 30%, 40%, 50%, 60%, 70%, 80%, 90%, 95% or more of the nucleotide or codon alterations are separated by at least about 3, 6, 9, 10, 12, 15, 25, 50, 100, or more nucleotides from the next closest nucleotide or codon alteration.

In an exemplary embodiment, the hierarchical assembly methods described herein may be used to produce a cell which has been codon remapped, e.g., a cell having a different pattern of codon usage as compared to a wild-type cell. In one embodiment, codon remapping refers to modifying the codon content of a nucleic acid sequence without modifying the sequence of the polypeptide encoded by the nucleic acid, e.g., via codon optimization, codon normalization, or codon reassignment. For example, a cell may have a genome that has been altered as compared to the wild-type genome but still maintains a wild-type proteome. In an exemplary embodiment, a cell may have all or substantially of the sequences of the essential genes in the genome altered as compared to a wild-type genome while all or substantially all of the polypeptide sequences encoded by the altered genome maintain the wild-type sequence.

Codon reassignment may be achieved, for example, by freeing up a degenerate codon and then reassigning the freed up codon to have a different specificity. A codon may be freed up by replacing all natural occurrences of a first codon in a genome with a second codon that is degenerate to the first codon. In certain embodiments, codon reassignment may be conducted using a modified tRNA and/or tRNA synthetase such that the cell inserts a non-wild-type amino acid in response to a codon, e.g., the cell inserts a naturally occurring amino acid in response to a codon that does not normally signal for that particular amino acid in a wild-type cell (e.g., a leucine is inserted in response to a codon that normal signals arginine or a translational stop, etc.). In other embodiments, a non-wild-type amino acid may be an unnatural amino acid. For example, codon reassignment may be conducted using a modified tRNA and/or tRNA synthetase that inserts an unnatural amino acid in response to a codon.

In one embodiment, the hierarchical assembly methods described herein may be used to codon remap at least a portion of all coding sequences in a genome (e.g., genes and essential genes). In other embodiments, at least a portion of all essential genes in a genome may be codon remapped using the methods provided herein.

In an exemplary embodiment, the hierarchincal assembly methods described herein may be used to produce a genome in which all original occurrences of a given codon have been removed from the genome, e.g., freeing up a codon by replacing all original occurrences of a first codon with one or more different codons degenerate thereto. For example, all original occurrences of a first degenerate codon such as a leucine codon or a stop codon may be removed from a genome by replacing the first leucine codon with a different leucine codon or the first stop codon with a different stop codon, respectively. In other embodiments, such a genome may be further modified so that it comprises a tRNA and/or tRNA synthetase that inserts a non-wild-type amino acid in response to the freed up codon (e.g., the freed up codon has been reassigned). Nucleic acid sequences containing one or more occurrences of the freed up, reassigned codon may then be introduced into the cell at new locations, e.g., at locations different from the original locations of the freed up codon in the genome. The sequences containing occurrences of the reassigned codon may be in the genome itself or may be maintained on an extrachromosomal nucleic acid, such as, for example, a plasmid. The sequences containing occurrences of the reassigned codon will lead to the expression of polypeptides wherein the non-wild-type amino acid has been incorporated into the polypeptide in response to the reassigned codon.

In an exemplary embodiment, the cell is modified so that at least one stop codon is freed up (e.g., all or substantially all of the original occurrences of one stop codon in the genome are removed by replacing the stop codon with a different stop codon). In certain embodiments, the cell may be further modified so that expression of the corresponding release factor (RF) is partially or completely reduced or inhibited in the host cell (e.g., by mutating or removing part or all of the nucleotide sequence encoding the release factor or sequences controlling expression of the release factor). The freed up stop codon may then be reassigned by introducing into the cell a tRNA and/or tRNA synthetase that inserts an amino acid in response to the freed up stop codon (e.g., the freed up stop codon has been reassigned). Nucleic acid sequences encoding polypeptides and containing occurrences of the freed up, reassigned stop codon may then be introduced into the cell and the amino acid charged by the tRNA that recognizes the stop codon will be introduced into the polypeptide chain in response to the freed up, reassigned stop codon. Partially or completely reducing the release factor facilitates readthrough of the translation machinery past the reassigned stop codon. In bacteria there are two release factors (RFs) referred to as RF1 and RF2 which recognize the UAA/UAG and UAA/UGA stop codons, respectively. In eukaryotes, archaea and mitochondria there is only a single release factor protein (eRF1, aRF1 and mtRF1, respectively) that recognizes all three stop codons (see e.g., Kisselev et al., EMBO J. 22: 175-182 (2003)). In exemplary embodiments, expression and/or activity of a release factor is partially or completely reduced or inhibited by introducing one or more one or more of the following types of mutations into the release factor sequence: point mutations; mutations that introduce at least one, two or more stop codons (other than the reassigned stop codon) into the open reading frame of the release factor gene; missense mutation in the open reading frame of the release factor gene; at least one mutation in a conserved and/or functionally important region of a release factor; mutations that are located in the open reading frame of the release factor in proximity to the translational start site; mutations in the transcriptional control sequences of the release factor gene; mutations in the translational control sequences of the release factor gene; and/or mutations that cause translation termination near the N-terminus of the release factor (e.g., within 50 amino acids, 25 amino acids, 10 amino acids, 5 amino acids, or less, of the N-terminus). In another embodiment, the entire coding sequence for the release factor polypeptide may be removed from the genome. In various embodiments, the cell in which expression and/or activity of a release factor is partially or completely reduced or inhibited may be, for example, a prokaryotic cell, such as a bacterial cell. In an exemplary embodiment, the cell is an E. coli cell.

In certain embodiments, codon remapping of a sequence may involve modification of the encoded polypeptide sequences. In such embodiments, the polypeptide sequences may be modified such that the amino acid changes are conservative.

A transposable genetic element is a genetic unit, such as a transposon, that can insert into, exit from, or relocate within a genome, chromosome, or plasmid. A transposon is a region of a genome that may be flanked by inverted repeats, direct repeats, or no significant repeats. A copy of the transposon can be inserted at a different location in the genetic material of an organism. Typically a transposon includes a gene encoding a transposase which is a protein that catalyzes the transposition of the genetic element, including the DNA encoding the transposase itself. A transposase may induce integration at random locations in some or all species in which it is operative, or it may insert the element at a specific site, and may behave differently in different species (see, e.g., Osborn et al, Plasmid 48: 202-212 (2002)). Transposable genetic elements are active in many microorganisms, and serve to protect the species from insertions, to relieve metabolic stresses, and to drive evolutionary change. Microbial expression vehicles, which are laboriously engineered to maximize expression of a gene or synthesis of a small molecule or polymer through a metabolic pathway, often lose their engineered phenotype through transposition of genetic elements. Thus, in a matter of a few generations, clones which have experienced a transposition event that inactivates overexpression, or otherwise serves to give the cell a metabolic advantage, or relieve metabolic stress, can swamp the culture.

In accordance with one aspect of the invention, a transposon knock down cell may be produced as an expression vehicle having improved phenotypic stability, or for other purposes. This is accomplished by intentionally mutating the DNA sequences encoding the open reading frams (ORFs) or control elements of transposase enzymes, preferably all copies of all transposase enzymes in the genome of a cell, so that they are inoperative. This inactivates DNA segment jumping within a cell and among cells, and reduces the frequency and speed of spontaneous reversion of carefully engineered cells to wild type characteristics. This may be done together with other efforts to modify the genome of an organism as discussed herein, or alone by genome-wide point mutations. Recombination into the genome of specially designed polynucleotide constructs is ideal for this purpose. In certain embodiments, the point mutations may be in a region that effects expression of the transposase, for example, in the coding regions, or a region that controls expression of the transposase. Alternatively, the point mutations may be made in regions flanking the coding region of the transposase that facilitate copying, excision, or insertion of the transposon element. For example, in a case where the transposon is flanked by inverted repeats, the point mutations may be made in the inverted repeats. Various combinations of point mutations in the transposase coding region, transposase transcriptional and/or translational control sequences, and/or flanking regions are also contemplated. In certain embodiments, a single transposon may be inactivated by introduction of one or more point mutations, including, 1, 2, 3, 4, 5, 6, 7, 8, 9, 10, or more, point mutations in a single transposon. In an exemplary embodiment, at least one point mutation that terminates translation of a transposase (e.g., a stop codon) is introduced into the DNA sequence such that translation of the protein is terminated near the N-terminus, e.g., within 2, 5, 10, 15, 20, 25, 30, or 50 amino acids of the N-terminus of the transposase. In yet another embodiment, two or more stop codons are introduced toward the 5′ end of the transposase coding sequence. In certain embodiments, the genome size and/or spacing is not changed upon introduction of the point mutations. In certain embodiments, the nucleotide modifications do not substantially change the overall size of the genome, for example, the size of the genome is reduced by less than 10%, 5%, 1%, 0.5%, 0.25%, 0.1%, 0.01% or less, or the genome is reduced by less than 500, 200, 100, 50, 25, 10, 5, or less base pairs. In an exemplary embodiment, the point mutations modify one or more bases to different bases but do not delete any bases from the genome (e.g., the genome size remains the same).

In yet other embodiments, mutations in protein and/or nucleic acid sequences involved in conjugation, mating, and/or transformation may be introduced. When combined with other genome modifications as described herein, significantly higher safety may be achieved.

The methods provided herein also permit construction of organisms having an altered genetic code. For example, the ability to make genome wide modifications permits the construction of organisms that utilize a different codon complement and/or a different tRNA complement as compared to a wild-type cell. Such organisms may be genetically isolated from wild-type organism because they cannot donate sequences that are properly expressed in wild-type cells and/or they cannot properly express sequences received from a wild-type organism. Additionally, genome wide modifications permit production of proteins that contain unnatural amino acids, e.g., by using altered tRNA molecules that are charged with an unnatural amino acid and will insert the amino acid in response to a desired codon. Additionally, it is possible to construct organisms that have an altered organelle genome (e.g., mitochondrial genome or chloroplast genome). It may be possible to genetically alter chromosomal DNA and/or extrachromosomal DNA in a given cell.

In various embodiments, all or a portion of a genome may be codon remapped, including, for example, (1) codon remapping of the entire genome, including both the chromosomal DNA as well as DNA contained in an organelle, such as, for example, a mitochondria or chloroplast, (2) codon remapping of only the chromosomal DNA but not any extrachromosomal DNA, or (3) codon remapping of only DNA contained in one more organelles, such as, for example, a mitochondria or a chloroplast, but not the chromosomal DNA. Certain organelles, including, for example, mitochondria contain their own complement of tRNAs that may vary as compared to the complement of cyoplasmic tRNAs. For example, in mitochondria a UGA codon encodes for a tryptophan residue as opposed to chain termination, in mammalian mitochondria an AUA codon specifies a methionine rather than an isoleucine, in vertebrate mitochondria an AGA or AGG codon specifies chain termination rather than an arginine, and in yeast mitochondria CUX codons specify threonine instead of leucine. Annotated complete DNA sequences are available for 213 mitochondrial genomes for 132 species (see e.g., Knight et al. J. Mol. Evol. 53: 299-313 (2001)). Methods for introducing DNA into mitochondria and/or modifying mitochondrial DNA may be made using methods known in the art based on the disclosures herein (see e.g., Knight et al., J. Mol. Evol. 53: 299-313 (2001), Geol et al., Nucleic Acids Res. 31: 1407-1415 (2003), and Barrell et al., Proc. Natl. Acad. Sci. 77: 3164-3166 (1980)).

In one exemplary embodiment, the invention includes a host cell in which new genes have been inserted into the mitochondrial DNA wherein the genes have been codon remapped for expression using the mitochondrial complement of tRNAs. For example, a gene not normally expressed in the mitochondria may be modified so that the same protein sequence will be expressed in the mitochondrial environment using the complement of tRNAs present in the mitochondria. In another exemplary embodiment, the invention provides for a cell in which the mitochondrial genome has been codon remapped and at least one modified tRNA has been introduced into the mitochondria (the corresponding wild-type tRNA may optionally be deleted or inactivated). The chromososmal DNA of the host cell may or may not be codon remapped. In this embodiment, the codon remapped mitochondria is isolated from receipt of wild-type mitochondrial sequences. In yet another exemplary embodiment, the invention provides a host cell in which the mitochondrial genome has been codon remapped, at least one modified tRNA has been introduced into the mitochondria (the corresponding wild-type tRNA may optionally be deleted or inactivated), and a nucleic acid which has been codon remapped is introduced into the host cell. The chromosomal DNA of the host cell may or may not be codon remapped. In this embodiment, the codon remapped mitochondria is isolated from receipt of wild-type mitochondrial sequences and also is isolated from donation of mitochondrial DNA to a wild-type mitochondria.

As will be apparent to the skilled genetic engineering, this specification enables the wholesale alteration of any part, or all, of an organism's genome, and this capability can be exploited to impart individually, or in various combinations, the utilities and features disclosed herein, and other features which are enabled by this capability. In certain embodiments, additional safeguards such as nutritional auxotrophies or required signals may be used to limit proliferation of the cells to only the intended environments.

5. Homologous Recombination/Site-specific Recombination

In certain embodiments, large polynucleotide constructs such as, for example, modified, partially synthetic, and/or fully synthetic genomes may be produced by replacing portions of the polynucleotide scaffold (e.g., genome or extrachromonsomal nucleic acid) with corresponding portions containing the desired sequence modifications. For example, this may be achieved by homologous recombination of long DNA molecules prepared as described above and containing the desired sequence substitutions. Alternatively, site-specific recombination using one or more integrases may be used to create a product nucleic acid, such as, for example, a modified, partially synthetic, and/or fully synthetic genome.

“Homologous recombination” is a process by which an exogenously introduced DNA molecule integrates into a target DNA molecule in a region where there is identical or near-identical nucleotide sequence between the two molecules. Homologous recombination is mediated by complementary base-pairing, and may result in either insertion of the exogenous DNA into the target DNA (a single cross-over event), or replacement of the target DNA by the exogenous DNA (a double cross-over event). Homologous recombination may occur in any cell having a “homologous recombination system”, e.g., one or more polypeptides that facilitate homologous recombination in a cell. Homologous recombination systems may be endogenous to the cell or may be introduced into the cell using recombinant technology. In an exemplary embodiment, a homologous recombination system refers to one or more of the exo, bet (β) and gam (γ) genes from phage lambda (λ). Homologous recombination may occur in virtually any cell type, including bacterial, archaebacterial, yeast, fungal, algal, plant, or animal (including mammalian and isolated human) cells.

Alternatively, in site-specific recombination, exchange occurs at a specific site, as in the integration of phage λ into the E. coli chromosome and the excision of λ DNA from the E. coli chromosome. Site-specific recombination involves specific (e.g., inverted repeat, non-repetitive, etc.) sequences; e.g. the Cre-loxP and FLP-FRT systems. Within these sequences there is only a short stretch of homology necessary for the recombination event, but not sufficient for it. The enzymes involved in this event generally cannot recombine other pairs of homologous (or nonhomologous) sequences, but act specifically.

Although both site-specific recombination and homologous recombination are useful mechanisms for genetic engineering of DNA sequences, targeted homologous recombination provides a basis for targeting and altering essentially any desired sequence in a duplex DNA molecule, such as targeting a DNA sequence in a chromosome for replacement by another sequence. Site-specific recombination has been proposed as one method to integrate transfected DNA at chromosomal locations having specific recognition sites (O'Gorman et al. (1991) Science 251: 1351; Onouchi et al. (1991) Nucleic Acids Res. 19: 6373). Since this approach requires the presence of specific target sequences and recombinases, its utility for targeting recombination events at any particular chromosomal location is limited in comparison to targeted general recombination. Mitigating this requirement is the availability of designed site-specific recombination initiators (see e.g., Urnov F D, et al. Nature 435: 646-651 (2005)).

A primary step in homologous recombination is DNA strand exchange, which involves a pairing of a DNA duplex with at least one DNA strand containing a complementary sequence to form an intermediate recombination structure containing heteroduplex DNA (see, Radding, C. M. (1982) Ann. Rev. Genet. 16: 405; U.S. Pat. No. 4,888,274). The heteroduplex DNA may take several forms, including a three DNA strand containing triplex form wherein a single complementary strand invades the DNA duplex (Hsieh et al. (1990) Genes and Development 4: 1951; Rao et al., (1991) PNAS 88:2984)) and, when two complementary DNA strands pair with a DNA duplex, a classical Holliday recombination joint or chi structure (Holliday, R. (1964) Genet. Res. 5: 282) may form, or a double-D loop. Once formed, a heteroduplex structure may be resolved by strand breakage and exchange, so that all or a portion of an invading DNA strand is spliced into a recipient DNA duplex, adding or replacing a segment of the recipient DNA duplex. Alternatively, a heteroduplex structure may result in gene conversion, wherein a sequence of an invading strand is transferred to a recipient DNA duplex by repair of mismatched bases using the invading strand as a template (Genes, 3rd Ed. (1987) Lewin, B., John Wiley, New York, N.Y.; Lopez et al. (1987) Nucleic Acids Res. 15: 5643). Whether by the mechanism of breakage and rejoining or by the mechanism(s) of gene conversion, formation of heteroduplex DNA at homologously paired joints can serve to transfer genetic sequence information from one DNA molecule to another.

The ability of homologous recombination (gene conversion and classical strand breakage/rejoining) to transfer genetic sequence information between DNA molecules makes targeted homologous recombination a powerful method in genetic engineering and gene manipulation.

The ability of cells to incorporate exogenous genetic material into genes residing on chromosomes has demonstrated that some cells (including yeast, mammals and humans) have the general enzymatic machinery for carrying out homologous recombination required between resident and introduced sequences. These targeted recombination events can be used to correct mutations at known sites, replace genes or gene segments with defective ones, or introduce foreign genes into cells. The efficiency of such gene targeting techniques is related to several parameters: the efficiency of DNA delivery into cells, the type of DNA packaging (if any) and the size and conformation of the incoming DNA, the length and position of regions homologous to the target site (all these parameters also likely affect the ability of the incoming homologous DNA sequences to survive intracellular nuclease attack), the efficiency of hybridization and recombination at particular chromosomal sites and whether recombinant events are homologous or nonhomologous.

Exogenous sequences transferred into eukaryotic cells undergo homologous recombination with homologous endogenous sequences only at very low frequencies, and are so inefficiently recombined that large numbers of cells must be transfected, selected, and screened in order to generate a desired correctly targeted homologous recombinant (Kucherlapati et al. (1984) Proc. Natl. Acad. Sci. (U.S.A.) 81: 3153; Smithies, O. (1985) Nature 317: 230; Song et al. (1987) Proc. Natl. Acad. Sci. (U.S.A.) 84: 6820; Doetschman et al. (1987) Nature 330: 576; Kim and Smithies (1988) Nucleic Acids Res. 16: 8887; Doetschman et al. (1988) op.cit.; Koller and Smithies (1989) op.cit.; Shesely et al. (1991) Proc. Natl. Acad. Sci. (U.S.A.) 88: 4294; Kim et al. (1991) Gene 103: 227, which are incorporated herein by reference).

Koller et al. (1991) Proc. Natl. Acad. Sci. (U.S.A.), 88: 10730 and Snouwaert et al. (1992) Science 257: 1083, have described targeting of the mouse cystic fibrosis transmembrane regulator (CFTR) gene for the purpose of inactivating, rather than correcting, a murine CFTR allele. Koller et al. employed a large (7.8 kb) homology region in the targeting construct, but nonetheless reported a low frequency for correct targeting (only 1 of 2500 G418-resistant cells were correctly targeted). Thus, even targeting constructs having long homology regions are inefficiently targeted.

Several proteins or purified extracts having the property of promoting homologous recombination (i.e., recombinase activity) have been identified in prokaryotes and eukaryotes (Cox and Lehman (1987) Ann. Rev. Biochem. 56: 229; Radding, C. M. (1982) op.cit.; Madiraju et al. (1988) Proc. Natl. Acad. Sci. (U.S.A.) 85: 6592; McCarthy et al. (1988) Proc. Natl. Acad. Sci. (U.S.A.) 85: 5854; Lopez et al. (1987) op.cit., which are incorporated herein by reference). These general recombinases presumably promote one or more steps in the formation of homologously-paired intermediates, strand-exchange, gene conversion, and/or other steps in the process of homologous recombination.

The frequency of homologous recombination in prokaryotes is significantly enhanced by the presence of recombinase activities. Several purified proteins catalyze homologous pairing and/or strand exchange in vitro, including: E. coli recA protein, the T4 uvsX protein, the rec1 protein from Ustilago maydis, and Rad51 protein from S. cervisiae (Sung et al., Science 265:1241 (1994)) and human cells (Baumann et al., Cell 87:757 (1996)). Recombinases, like the recA protein of E. coli are proteins which promote strand pairing and exchange. The most studied recombinase to date has been the recA recombinase of E. coli, which is involved in homology search and strand exchange reactions (see, Cox and Lehman (1987), supra). RecA is required for induction of the SOS repair response, DNA repair, and efficient genetic recombination in E. coli. RecA can catalyze homologous pairing of a linear duplex DNA and a homologous single strand DNA in vitro. In contrast to site-specific recombinases, proteins like recA which are involved in general recombination recognize and promote pairing of DNA structures on the basis of shared homology, as has been shown by several in vitro experiments (Hsieh and Camerini-Otero (1989) J. Biol. Chem. 264: 5089; Howard-Flanders et al. (1984) Nature 309: 215; Stasiak et al. (1984) Cold Spring Harbor Symp. Quant. Biol. 49: 561; Register et al. (1987) J. Biol. Chem. 262: 12812). Several investigators have used recA protein in vitro to promote homologously paired triplex DNA (Cheng et al. (1988) J. Biol. Chem. 263: 15110; Ferrin and Camerini-Otero (1991) Science 354: 1494; Ramdas et al. (1989) J. Biol. Chem. 264: 11395; Strobel et al. (1991) Science 254: 1639; Hsieh et al. (1990) op.cit.; Rigas et al. (1986) Proc. Natl. Acad. Sci. (U.S.A.) 83: 9591; and Camerini-Otero et al. U.S. Pat. No. 7,611,268 (available from Derwent), which are incorporated herein by reference).

Common mechanisms for inducing recombination include, but are not limited to the use of strains comprising mutations such as those involved in mismatch repair. e.g. mutations in mutS, mutT, mutL and mutH; exposure to U.V. light; Chemical mutagenesis, e.g. use of inhibitors of MMR, DNA damage inducible genes, or SOS inducers; overproduction/underproduction/mutation of any component of the homologous recombination complex/pathway, e.g. RecA, ssb, etc.; overproduction/underproduction/mutation of genes involved in DNA synthesis/homeostasis; overproduction/underproduction/mutation of recombination-stimulating genes from bacteria, phage (e.g. Lambda Red function), or other organisms; addition of chi sites into/flanking the donor DNA fragments; coating the DNA fragments with RecA/ssb and the like.

In certain embodiments, the host cell may naturally be capable of carrying out homologous recombination. Recombination generally occurs through the activity of one or more polypeptides which form a recombination system. In some embodiments the host cell may contain an endogenous recombination system. In other embodiments, the host cell may contain an endogenous recombination system that may be enhanced by one or more exogenous factors that facilitate recombination in the host cell. For example, the host cell may be engineered to express a polypeptide involved in recombination or a recombination facilitating factor may be mixed with a polynucleotide construct prior to its introduction into the host cell. In still other embodiments, the host cell may be engineered to comprise a homologous recombination system that is not endogenous to the cell.

In an exemplary embodiment, a host cell comprises a recombination system having one or more polypeptides encoded by the genes selected from the group consisting of the exo, bet and gam genes from phage λ. The gam gene (also referred to as gamma or γ) encodes a protein which inhibits the RecBCD nuclease from degrading linear DNA while the exo and bet (also referred to as beta or β) genes encode proteins involved in homologous recombination. In one embodiment, the homologous recombination system is the phage X recombinase system comprising the exo, bet and gam genes of phage λ. Still other suitable recombination systems will be known to one of skill in the art.

The stuffer fragment of lambda 1059 carries the lambda exo, beta, gamma under the control of the leftward promoter (pL). These genes confer an Spi⁺ phenotype, i.e., the phage is able to grow on recA⁻ strains but is unable to grow on strains that are lysogenic for bacteriophage P2. Since pL is also located on the stuffer fragment, the expression of the Spi⁺ phenotype is not affected by the orientation of the stuffer between the left and right arms of the vector.

Wild-type members of the Enterobacteriaceae (e.g., Escherichia coli) are typically resistant to genetic exchange following transformation of linear DNA molecules. This is due, at least in part, to the Exonuclease V (Exo V) activity of the RecBCD holoenzyme which rapidly degrades linear DNA molecules following transformation. Production of ExoV has been traced to the recD gene, which encodes the D subunit of the holoenzyme. The RecBCD holoenzyme plays an important role in initiation of RecA-dependent homologous recombination. Upon recognizing a dsDNA end, the RecBCD enzyme unwinds and degrades the DNA asymmetrically in a 5′ to 3′ direction until it encounters a chi (or “χ”)-site (consensus 5′-GCTGGTGG-3′) which attenuates the nuclease activity. This results in the generation of a ssDNA terminating near the chi site with a 3′-ssDNA tail that is preferred for RecA loading and subsequent invasion of dsDNA for homologous recombination. Accordingly, preprocessing of transforming fragments with a 5′ to 3′ specific ssDNA Exonuclease, such as Lambda (λ) exonuclease (available, e.g., from Boeringer Mannheim) prior to transformation may serve to stimulate homologous recombination in recD⁻ strains by providing ssDNA invasive end for RecA loading and subsequent strand invasion.

The addition of sequences encoding chi-sites (consensus 5′-GCTGGTGG-3′) to DNA fragments can serve to both attenuate Exonuclease V activity and stimulate homologous recombination, thereby obviating the need for a recD mutation (see also, Kowalczykowski, et al. (1994) Microbiol. Rev. 58: 401-465; Jessen, et al. (1998) Proc. Natl. Acad. Sci. 95: 5121-5126).

In certain embodiments, chi-sites may be included in the polynucleotide constructs and/or segments described herein for assembly of large polynucleotide constructs. The use of recombination-stimulatory sequences such as chi is a generally useful approach for increasing the efficiency of homologous recombination in a wide variety of cell types.

Methods to inhibit or mutate analogs of Exo V or other nucleases (such as, Exonucleases I (endA1), III (nth), IV (nfo), VII, and VIII of E. coli) is similarly useful. Inhibition or elimination of such nucleases, or modification of ends of transforming DNA fragments to render them resistant to exonuclease activity has applications in facilitating homologous recombination in a broad range of cell types.

In certain embodiments, a homologous recombination system may comprise one or more endogenous and/or exogenous recombinase proteins. Recombinases are proteins that may provide a measurable increase in the recombination frequency and/or localization frequency between a targeting polynucleotide and a desired target sequence. The most common recombinase is a family of RecA-like recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to complementary endogenous sequences. The best characterized recA protein is from E. coli, in addition to the wild-type protein a number of mutant recA-like proteins have been identified (e.g., recA803). Further, many organisms have recA-like recombinases with strand-transfer activities (e.g., Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. USA 85: 36-40; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimiec, (1984) Cold Spring Harbor Symp. 48:675; Kimeic, (1986) Cell 44: 545; Kolodner et al., (1987) Proc. Natl. Acad. Sci. USA 84:5560; Sugino et al., (1985) Proc. Natl. Acad, Sci. USA 85: 3683; Halbrook et al., (1989) J. Biol. Chem. 264: 21403; Eisen et al., (1988) Proc. Natl. Acad. Sci. USA 85: 7481; McCarthy et al., (1988) Proc. Natl. Acad. Sci. USA 85: 5854; Lowenhaupt et al., (1989) J. Biol. Chem. 264: 20568). Examples of such recombinase proteins include, for example, but are not limited to: recA, recA803, uvsX, and other recA mutants and recA-like recombinases (Roca, A. I. (1990) Crit. Rev. Biochem. Molec. Biol. 25: 415), sepl (Kolodner et al. (1987) Proc. Natl. Acad. Sci. (U.S.A.) 84: 5560; Tishkoff et al. Molec. Cell. Biol. 11: 2593), RuvC (Dunderdale et al. (1991) Nature 354: 506), DST2, KEM1, XRN1 (Dykstra et al. (1991) Molec. Cell. Biol. 11: 2583), STP-alpha/DST1 (Clark et al. (1991) Molec. Cell. Biol. 11: 2576), HPP-1 (Moore et al. (1991) Proc. Natl. Acad. Sci. (U.S.A.) 88: 9067), other eukaryotic recombinases (Bishop et al. (1992) Cell 69: 439; Shinohara et al. (1992) Cell 69: 457). RecA may be purified from E. coli strains, such as E. coli strains JC12772 and JC15369 (available from A. J. Clark and M. Madiraju, University of California-Berkeley). These strains contain the recA coding sequences on a “runaway” replicating plasmid vector present at a high copy numbers per cell. The recA803 protein is a high-activity mutant of wild-type recA. The art teaches several examples of recombinase proteins, for example, from Drosophila, yeast, plant, human, and non-human mammalian cells, including proteins with biological properties similar to recA (i.e., recA-like recombinases).

In certain embodiments, recombinase protein(s) (prokaryotic or eukaryotic) may be exogenously administered to a host cell. Such administration is typically done by microinjection, although electroporation, lipofection, and other transfection methods known in the art may also be used. Alternatively, recombinase proteins may be produced in vivo from a heterologous expression cassette in a transfected cell or transgenic cell, such as a transgenic totipotent embryonal stem cell (e.g., a murine ES cell such as AB-1) used to generate a transgenic non-human animal line or a pluripotent hematopoietic stem cell for reconstituting all or part of the hematopoietic stem cell population of an individual. In exemplary embodiments, a heterologous expression cassette may include a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible, or other modulatable promoter construct so that expression of at least one species of recombinase protein from the cassette can by modulated for transiently producing recombinase(s) in vivo simultaneous or contemporaneous with introduction of a targeting polynucleotide into the cell. When a hormone-inducible promoter-enhancer combination is used, the cell must have the required hormone receptor present, either naturally or as a consequence of expression a co-transfected expression vector encoding such receptor.

For making transgenic non-human animals (which include homologously targeted non-human animals) embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 27-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germ line transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In another embodiment, site-specific recombination using one or more site-specific recombinases may be used to create a large polynucleotide construct such as a modified, partially synthetic, and/or fully synthetic genome. A site-specific recombinase refers to a type of recombinase which typically has at least the following four activities (or combinations thereof): (1) recognition of one or two specific nucleic acid sequences; (2) cleavage of said sequence or sequences; (3) topoisomerase activity involved in strand exchange; and (4) ligase activity to reseal the cleaved strands of nucleic acid. See Sauer, B., Current Opinions in Biotechnology 5:521-527 (1994). The strand exchange mechanism involves the cleavage and rejoining of specific DNA sequences in the absence of DNA synthesis (Landy, A. (1989) Ann. Rev. Biochem. 58:913-949). Examples of site-specific recombinases include the integrase family of proteins and the tyrosine recombinase family of proteins (See e.g., Esposito and Scocca, Nucleic Acids Research 25: 3605-3614 (1997); Nunes-Duby et al., Nucleic acids Research 26: 391-406 (1998); and U.S. Patent Application Publication Nos. 2003/0124555 and 2003/0077804). In certain embodiments, a site-specific recombinase may naturally be present in the cell which is to be used for assembly, for example, when the cell is a bacterial cell or a yeast cell. In such an embodiment, a nucleic acid molecule may be introduced into the cell and the endogenous site-specific recombinase will catalyze integration of the polynucleotide construct into the appropriate location in the nucleic acid scaffold, such as a genome. Alternatively, when the cell to be used for assembly does not naturally contain a site-specific recombinase, a nucleic acid encoding the recombinase may introduced into the cell along with (either simultaneously or sequentially) the polynucleotide construct to be inserted into the nucleic acid scaffold, such as a genome. The coding sequence for the recombinase may be integrated into the genome of the host cell, or may be maintained on an extrachromosomal nucleic acid (e.g., a plasmid) either stably or transiently.

In an exemplary embodiment, the site-specific recombinase is a tyrosine recombinase that targets insertion of a nucleic acid to a tRNA gene. Such tyrosine recombinases will be useful for modifying the tRNA complement in a modified, partially synthetic, and/or fully synthetic genomes, for example, by deleting or inactivating a wild-type tRNA, by modifying an endogenous tRNA (e.g., by modifying the sequence of the tRNA gene, such as, for example, the sequence of the anticodon loop, D loop, variable loop, TΨC loop, acceptor, etc.), and/or by replacing a wild-type tRNA gene with a modified tRNA gene. Examples of tyrosine recombinases that target nucleic acid insertion to a tRNA gene include, for example, HP1 (Cell 89: 227-37 (1997)), L5 (J. Bact. 181: 454-61 (1999)), DLP12 (J. Bacteriol. 171: 6197-205 (1989)), P4 (J. Mol. Biol. 196: 487-96 (1987)), P22 (J. Biol. Chem. 260: 4468-77 (1985)), P2 (J. Bacteriol. 175: 1239-49 (1993)), P186 (J. Mol. Biol. 191: 199-209 (1986)), phiR73 (J. Bacteriol. 173: 4171-81)), RP3 (Nucl. Acids Res. 23: 58-63 (1995)), phiCTX (Mol. Gen. Genet. 246:72-79 (1995)), MV4 (J. Bacteriol. 179: 1837-45 (1997)), SSV1 (Mol. Gen. Genet. 237: 334-42 (1993)), T12 (Mol. Microb. 23: 719-28 (1997)), A2 (Virology 250: 185-93 (1998)), PPu orf (J. Bacteriol. 180: 5505-14 (1998)), phi10MC (FEMS Microb. Let. 147: 279-85 (1997)), VWB (Microbiology 144: 3351-58 (1998)), and YPe orf (Mol. Microb. 31(1): 291-303 (1999)).

6. Modified tRNAs and tRNA Synthetases

The methods described herein may be used to produce codon remapped genomes that express a modified tRNA which inserts an amino acid upon exposure to a given codon that is not normally encoded by that codon. The amino acid may be a naturally occurring amino acid that is not normally associated with a given codon or the amino acid may be an unnatural amino acid. In an exemplary embodiment, the modified tRNA may be produced simply by changing the anticodon portion of the tRNA molecule itself. This modified tRNA molecule will still be charged with the same natural amino acid but will now insert this amino acid into a polypeptide chain in response to a different codon sequence. This type of modified tRNA may be constructed when the native tRNA synthetase that loads the tRNA does not interact or interacts minimally with the anticodon region during the loading process. In other embodiments, the tRNA synthetase may interact with the anticodon region of the tRNA molecule. In this case, a corresponding tRNA synthetase that is capable of loading the desired amino acid onto the tRNA must also be provided. Various references disclose methods for creating orthogonal tRNA/tRNA synthetase pairs that charge a desired amino acid onto a tRNA that recognizes a specific codon sequence. To date, over 100 noncoded amino acids (all ribosomally acceptable) have been reportedly introduced into proteins using various methods (see, for example, Schultz et al., J. Am. Chem. Soc., 103: 1563-1567, 1981; Hinsberg et al., J. Am. Chem. Soc., 104: 766-773, 1982; Pollack et al., Science, 242: 1038-1040, 1988; Nowak et al., Science, 268: 439-442, 1995; U.S. Patent Application Publication Nos. 2004/0053390; 2003/0143558; and 2003/0108885). These methods may be used for designing suitable altered amino acid tRNA synthetases (AARSs) that can efficiently charge a tRNA having a given anticodon with a desired amino acid.

A database of known tRNA genes and their sequences from a variety of organisms is publicly available (see e.g., http://rna.wustl.edu/GtRDB/). Similarly, a database of known aminoacyl tRNA synthetases (aaRSs) has been published by Maciej Szymanski, Marzanna A. Deniziak and Jan Barciszewski, in Nucleic Acids Res. 29:288-290, 2001 (titled “Aminoacyl-tRNA synthetases database”). A corresponding website (http://rose.man.poznan.pl/aars/seq_main.html) provides details about all known aaRSs from different species. For example, according to the database, the Isoleucyl-tRNA Synthetase for the radioresistant bacteria Deinococcus radiodurans (Accession No. AAF10907) has 1078 amino acids, and was published by White et al. in Science 286:1571-1577(1999); the Valyl-tRNA Synthetase for mouse (Mus musculus) has 1263 amino acids (Accession No. AAD26531), and was published by Snoek M. and van Vugt H. in Immunogenetics 49: 468-470(1999); and the Phenylalanyl-tRNA Synthetase sequences for human, Drosophila, S. pombe, S. cerevisiae, Candida albicans, E. coli, and mumerous other bacteria including Thermus aquaticus ssp. thermophilus are also available. Similar information for other newly identified aaRSs can be obtained, for example, by conducting a BLAST search using any of the known sequences in the aaRS database as query against the available public (such as the non-redundant database at NCBI, or “nr”) or proprietary private databases.

Modifications of tRNA have been demonstrated to play several key roles in maintaining the tRNA's ability to faithfully decode an mRNA sequence (see, e.g., Qian Q., et al., (1998) J Bacteriol 180(7):1808-13; Grosjean, H., et al., (1995) Biochimie 77:3-6; Esberg, B., et al., (1995) J Bacteriol 177(8):1967-75; and Grosjean, H., et al., (1998) “Modification and Editing of RNA,” American Society for Microbiology, Washington D.C., pp. 493-516). Furthermore, tRNA modifications have been implicated in full and proper translation of virulence genes in Shigella flexneri (see, e.g., Durand, J., et al., (1994) J Bacteriol 176(15):4627-34; Durand, J., et al., (1997) J Bacteriol 179(18):5777-82; and Durand, J., et al., (2000) Mol Microbiol 35(4):924-35) and in the plant pathogen Agrobacterium tumefaciens (see, e.g., Gray, J., et al. (1992) J Bacteriol 174(4):1086-9823). Modifications found at position 34 of the anticodon have been shown to change the coding capabilities of a particular tRNA by expanding or restricting the wobble rules at that position. For example, queosine (Q) replaces a guanosine at position 34 in Tyr, His, Asp, and Asn tRNAs and helps prevent misreading of the TAA/TAG STOP codons, and may prevent misreading of Gln, Lys, and Glu codons by restricting wobble. Alternatively, the lysidine modification at position 34 of the rare bacterial ileX tRNA changes its coding capacities from AUG to AUA (see, e.g., Muramatsu, T. et al., (1988) Nature 336(6195):179-81). Furthermore, modifications adjacent to the anticodon at position 37, including i6A and t6A, have been demonstrated to effect strand slipping and stop codon read through (see, e.g., Qian, Q., et al., (1998) J Bacteriol 180(7):1808-13; Esberg, B., et al., (1995) J Bacteriol 177(8):1967-75; and Miller, J., et al., (1976) Nuc Acids Res 3(5): 1185-201) and effects the fidelity of codon/anticodon interactions.

In certain embodiments, it may be desirable to utilize a modified tRNA, a modified tRNA synthetase, or a modified tRNA/tRNA synthetase pair. One of skill in the art will be able to produce such molecules based on the teachings herein. Modified tRNAs, tRNA synthetases and pairs thereof may be used to charge a tRNA having a given anticodon with a desired amino acid (either natural or unnatural). See e.g., U.S. Patent Application Publication No. 2003/0108885.

Methods for producing a modified tRNA synthetase are based on generating a pool of mutant synthetases from the framework of a wild-type synthetase, and then selecting for mutated tRNA synthetases (RSs) based on their specificity, for example, for a non-wild-type amino acid or an unnatural amino acid relative to the common twenty. The modified synthetase may be produced by mutating the synthetase, e.g., at the active site in the synthetase, at the editing mechanism site in the synthetase, at different sites by combining different domains of synthetases, or the like, and applying a positive and/or negative selection process (see e.g., U.S. Patent Application Publication No. 2003/0108885). In positive selection, suppression of a selector codon introduced at a nonessential position(s) of a positive marker allows cells to survive under positive selection pressure. In the presence of both natural and unnatural amino acids, survivors thus encode active synthetases charging the orthogonal suppressor tRNA with either a natural or unnatural amino acid. In the negative selection, suppression of a selector codon introduced at a nonessential position(s) of a negative marker removes synthetases with natural amino acid specificities. Survivors of the negative and positive selection encode synthetases that aminoacylate (charge) the orthogonal suppressor tRNA with unnatural amino acids only. These synthetases can then be subjected to further mutagenesis, e.g., DNA shuffling or other recursive mutagenesis methods. Of course, in other embodiments, the invention optionally can utilize different orders of steps to identify (e.g., RS, tRNA, pairs, etc.), e.g., negative selection/screening followed by positive selection/screening or vice verse or any such combinations thereof.

For example, a selector codon, e.g., an amber codon, is placed in a reporter gene, e.g., an antibiotic resistance gene, such as β-lactamase, with a selector codon, e.g., TAG. This is placed in an expression vector with members of the mutated RS library. This expression vector along with an expression vector with a target tRNA, e.g., a suppressor tRNA, are introduced into a cell, which is grown in the presence of a selection agent, e.g., antibiotic media, such as ampicillin. Only if the synthetase is capable of aminoacylating (charging) the suppressor tRNA with some amino acid does the selector codon get decoded allowing survival of the cell on antibiotic media.

Applying this selection in the presence of the unnatural amino acid, the synthetase genes that encode synthetases that have some ability to aminoacylate are selected away from those synthetases that have no activity. The resulting pool of synthetases can be charging any of the 20 naturally occurring amino acids or the unnatural amino acid. To further select for those synthetases that exclusively charge the unnatural amino acid, a second selection, e.g., a negative selection, is applied. In this case, an expression vector containing a negative selection marker and a target tRNA is used, along with an expression vector containing a member of the mutated RS library. This negative selection marker contains at least one selector codon, e.g., TAG. These expression vectors are introduced into another cell and grown without unnatural amino acids and, optionally, a selection agent, e.g., tetracycline. In the negative selection, those synthetases with specificities for natural amino acids charge the orthogonal tRNA, resulting in suppression of a selector codon in the negative marker and cell death. Since no unnatural amino acid is added, synthetases with specificities for the unnatural amino acid survive. For example, a selector codon, e.g., a stop codon, is introduced into the reporter gene, e.g., a gene that encodes a toxic protein, such as barnase. If the synthetase is able to charge the suppressor tRNA in the absence of unnatural amino acid, the cell will be killed by translating the toxic gene product. Survivors passing both selection/screens encode synthetases specifically charging the orthogonal tRNA with an unnatural amino acid.

In one embodiment, methods for producing at least one recombinant modified aminoacyl-tRNA synthetase (RS) include: (a) generating a library of mutant RSs derived from at least one aminoacyl-tRNA synthetase (RS) from a first organism; (b) selecting the library of mutant RSs for members that aminoacylate a target tRNA in the presence of an unnatural amino acid and a natural amino acid, thereby providing a pool of active mutant RSs; and, (c) negatively selecting the pool for active mutant RSs that preferentially aminoacylate the target tRNA in the absence of the unnatural amino acid, thereby providing the at least one modified recombinant RS; wherein the at least one recombinant modified RS preferentially aminoacylates the target tRNA with the unnatural amino acid. Optionally, more mutations are introduced by mutagenesis, e.g., random mutagenesis, recombination or the like, into the selected synthetase genes to generate a second-generation synthetase library, which is used for further rounds of selection until a mutant synthetase with desired activity is evolved. Recombinant modified RSs produced by the methods are included in the present invention.

The library of mutant RSs can be generated using various mutagenesis techniques known in the art. For example, the mutant RSs can be generated by site-specific mutations, random point mutations, in vitro homologous recombinant, chimeric constructs or the like. In one embodiment, mutations are introduced into the editing site of the synthetase to hamper the editing mechanism and/or to alter substrate specificity. Libraries of mutant RSs also include chimeric synthetase libraries, e.g., libraries of chimeric Methanococcus jannaschii/Escherichia coli synthetases. The domain of one synthetase can be added or exchanged with a domain from another synthetase, such as, for example, the CPI domain. See, e.g., Sieber, et al., Nature Biotechnology, 19:456460 (2001). The chimeric library is screened for a variety of properties, e.g., for members that are expressed and in frame, for members that lack activity with a desired synthetase, and/or for members that show activity with a desired synthetase.

In one embodiment, the positive selection step includes: introducing a positive selection marker, e.g., an antibiotic resistance gene, or the like, and the library of mutant RSs into a plurality of cells, wherein the positive selection marker comprises at least one selector codon, e.g., an amber codon; growing the plurality of cells in the presence of a selection agent; selecting cells that survive in the presence of the selection agent by suppressing the at least one selector codon in the positive selection marker, thereby providing a subset of positively selected cells that contains the pool of active mutant RSs. Optionally, the selection agent concentration can be varied.

In one embodiment, negative selection includes: introducing a negative selection marker with the pool of active mutant RSs from the positive selection into a plurality of cells of a second organism, wherein the negative selection marker is an antibiotic resistance gene, e.g., a chloramphenicol acetyltransferase (CAT) gene, comprising at least one selector codon; and, selecting cells that survive in a 1st media supplemented with the unnatural amino acid and a selection agent, but fail to survive in a 2nd media not supplemented with the unnatural amino acid and the selection agent, thereby providing surviving cells with the at least one recombinant modified RS. Optionally, the concentration of the selection agent is varied.

In another embodiment, negatively selecting the pool for active mutant RSs includes: isolating the pool of active mutant RSs from the positive selection step (b); introducing a negative selection marker, wherein the negative selection marker is a toxic marker gene, e.g., a ribonuclease barnase gene, comprising at least one selector codon, and the pool of active mutant RSs into a plurality of cells of a second organism; and selecting cells that survive in a 1 st media not supplemented with the unnatural amino acid, but fail to survive in a 2nd media supplemented with the unnatural amino acid, thereby providing surviving cells with the at least one recombinant modified RS, wherein the at least one recombinant modified RS is specific for the unnatural amino acid. Optionally, the negative selection marker comprises two or more selector codons.

In one embodiment, positive selection is based on suppression of a selector codon in a positive selection marker, e.g., a chloramphenicol acetyltransferase (CAT) gene comprising a selector codon, e.g., an amber stop codon, in the CAT gene, so that chloramphenicol can be applied as the positive selection pressure. In addition, the CAT gene can be used as both a positive marker and negative marker as describe herein in the presence and absence of an unnatural amino acid. Optionally, the CAT gene comprising a selector codon is used for the positive selection and a negative selection marker, e.g., a toxic marker, such as a barnase gene comprising at least one or more selector codons, is used for the negative selection.

In another embodiment, positive selection is based on suppression of a selector codon at nonessential position in the β-lactamase gene, rendering cells ampicillin resistant; and a negative selection using the ribonuclease barnase as the negative marker is used. In contrast to β-lactamase, which is secreted into the periplasm, CAT localizes in the cytoplasm; moreover, ampicillin is bacteriocidal, while chloramphenicol is bacteriostatic.

The recombinant modified RS can be further mutated and selected. In one embodiment, the methods for producing at least one recombinant modified aminoacyl-tRNA synthetase can further comprise: (d) isolating the at least one recombinant modified RS; (e) generating a second set of mutated RS derived from the at least one recombinant modified RS; and, (f) repeating steps (b) and (c) until a mutated RS is obtained that comprises an ability to preferentially aminoacylate the target tRNA. Optionally, steps (d)-(f) are repeated, e.g., at least about two times. In one embodiment, the second set of mutated RS can be generated by mutagenesis, e.g., random mutagenesis, site-specific mutagenesis, recombination or a combination thereof.

The stringency of the selection steps, e.g., the positive selection step (b), the negative selection step (c), or both the positive and negative selection steps (b) and (c), in the above described-methods, optionally include varying the selection stringency. For example, because bamase is an extremely toxic protein, the stringency of the negative selection can be controlled by introducing different numbers of selector codons into the barnase gene. The stringency may be varied because the desired activity can be low during early rounds. Thus, less stringent selection criteria are applied in early rounds and more stringent criteria are applied in later rounds of selection.

Other types of selections can be used in the present invention for, e.g., modified RS, modified tRNA, and modified tRNA/RS pairs. For example, the positive selection step

(b), the negative selection step (c), or both the positive and negative selection steps (b) and (c), can include using a reporter, wherein the reporter is detected by fluorescence-activated cell sorting (FACS). For example, a positive selection can be done first with a positive selection marker, e.g., chloramphenicol acetyltransferase (CAT) gene, where the CAT gene comprises a selector codon, e.g., an amber stop codon, in the CAT gene, which followed by a negative selection screen, that is based on the inability to suppress a selector codon(s), e.g., two or more, at positions within a negative marker, e.g., T7 RNA polymerase gene. In one embodiment, the positive selection marker and the negative selection marker can be found on the same vector, e.g., plasmid. Expression of the negative marker drives expression of the reporter, e.g., green fluorescent protein (GFP). The stringency of the selection and screen can be varied, e.g., the intensity of the light need to fluorescence the reporter can be varied. In another embodiment, a positive selection can be done with a reporter as a positive selection marker, which is screened by FACs, followed by a negative selection screen, that is based on the inability to suppress a selector codon(s), e.g., two or more, at positions within a negative marker, e.g., barnase gene.

Methods for producing a modified tRNA are also provided. For example, to change the codon specificity of the tRNA while preserving its affinity toward a desired RS, the methods include a combination of negative and positive selections with a mutant suppressor tRNA library in the absence and presence of the cognate synthetase, respectively. In the negative selection, a selector codon(s) is introduced in a marker gene, e.g., a toxic gene, such as barnase, at a nonessential position. When a member of the mutated tRNA library, e.g., derived from Methanococcus jannaschii, is aminoacylated by endogenous host, e.g., Escherichia coli synthetases (i.e., it is not orthogonal to the host, e.g., Escherichia coli synthetases), the selector codon, e.g., an amber codon, is suppressed and the toxic gene product produced leads to cell death. Cells harboring modified tRNAs or non-functional tRNAs survive. Survivors are then subjected to a positive selection in which a selector codon, e.g., an amber codon, is placed in a positive marker gene, e.g., a drug resistance gene, such a β-lactamase gene. These cells also contain an expression vector with a cognate RS. These cells are grown in the presence of a selection agent, e.g., ampicillin. tRNAs are then selected for their ability to be aminoacylated by the coexpressed cognate synthetase and to insert an amino acid in response to this selector codon. Cells harboring non-functional tRNAs, or tRNAs that cannot be recognized by the synthetase of interest are sensitive to the antibiotic. Therefore, tRNAs that: (i) are not substrates for endogenous host, e.g., Escherichia coli, synthetases; (ii) can be aminoacylated by the synthetase of interest; and (iii) are functional in translation survive both selections.

Methods of producing a modified tRNA include: (a) generating a library of mutant tRNAs derived from at least one tRNA, e.g., a suppressor tRNA, from a first organism; (b) negatively selecting the library for mutant tRNAs that are aminoacylated by an aminoacyl-tRNA synthetase (RS) from a second organism in the absence of a RS from the first organism, thereby providing a pool of mutant tRNAs; and, (c) selecting the pool of mutant tRNAs for members that are aminoacylated by an introduced RS, thereby providing at least one modified tRNA; wherein the at least one recombinant modified tRNA recognizes a selector codon and is not efficiently recognized by the RS from the second organism and is preferentially aminoacylated by the RS.

Libraries of mutated tRNA may be constructed. For example, mutations can be introduced at a specific position(s), e.g., at a nonconservative position(s), or at a conservative position, at a randomized position(s), or a combination of both in a desired loop of a tRNA, e.g., an anticodon loop, (D arm, V loop, T.psi.C arm) or a combination of loops or all loops. Chimeric libraries of tRNA are also included in the present invention. It should be noted that libraries of tRNA synthetases from various organism (e.g., microorganisms such as eubacteria or archaebacteria) such as libraries comprising natural diversity (such as libraries that comprise natural diversity (see, e.g., U.S. Pat. No. 6,238,884 to Short et al. and references therein, U.S. Pat. No. 5,756,316 to Schallenberger et al; U.S. Pat. No. 5,783,431 to Petersen et al; U.S. Pat. No. 5,824,485 to Thompson et al; and U.S. Pat. No. 5,958,672 to Short et al), are optionally constructed and screened for orthogonal pairs.

In one embodiment, negatively selecting the library for mutant tRNAs that are aminoacylated by an aminoacyl-tRNA synthetase (step (b) above) includes: introducing a toxic marker gene, wherein the toxic marker gene comprises at least one of the selector codons and the library of mutant tRNAs into a plurality of cells from the second organism; and, selecting surviving cells, wherein the surviving cells contain the pool of mutant tRNAs comprising at least one orthogonal tRNA or nonfunctional tRNA. For example, the toxic marker gene is optionally a ribonuclease barnase gene, wherein the ribonuclease barnase gene comprises at least one amber codon. Optionally, the ribonuclease barnase gene can include two or more amber codons. The surviving cells can be selected, e.g., by using a comparison ratio cell density assay.

In one embodiment, selecting the pool of mutant tRNAs for members that are aminoacylated by an introduced RS can include: introducing a positive selection marker gene, wherein the positive selection marker gene comprises a drug resistance gene, e.g., a β-lactamase gene, comprising at least one of the selector codons, e.g., a β-lactamase gene comprising at least one amber stop codon, the RS, and the pool of mutant tRNAs into a plurality of cells from the second organism; and, selecting surviving cells grown in the presence of a selection agent, e.g., an antibiotic, thereby providing a pool of cells possessing the at least one recombinant tRNA, wherein the recombinant tRNA is aminoacylated by the RS and inserts an amino acid into a translation product encoded by the positive marker gene, in response to the at least one selector codons. In another embodiment, the concentration of the selection agent is varied.

As described above for generating modified RS, the stringency of the selection steps can be varied. In addition, other selection/screening procedures, which are described herein, such as FACs, cell and phage display can also be used.

Methods for producing a recombinant modified tRNA include: (a) generating a library of mutant tRNAs derived from at least one tRNA, e.g., a suppressor tRNA, from a first organism; (b) selecting (e.g., negatively selecting) or screening the library for (optionally mutant) tRNAs that are aminoacylated by an aminoacyl-tRNA synthetase (RS) from a second organism in the absence of a RS from the first organism, thereby providing a pool of tRNAs (optionally mutant); and, (c) selecting or screening the pool of tRNAs (optionally mutant) for members that are aminoacylated by an introduced RS, thereby providing at least one recombinant modified tRNA; wherein the at least one recombinant modified tRNA recognizes a selector codon and is not efficiently recognized by the RS from the second organism and is preferentially aminoacylated by the RS. In some embodiments, the modified tRNA is optionally imported into a first organism from a second organism without the need for sequence modifications. In various embodiments, the first and second organisms are either the same or different and are optionally chosen from, e.g., prokaryotes (e.g., Methanococcus jannaschii, Methanobacteium thermoautotrophicum, Escherichia coli, Halobacterium, etc.), eukaryotes, mammals, fungi, yeasts, archaebacteria, eubacteria, plants, insects, protists, etc. Additionally, the recombinant tRNA is optionally aminoacylated by an unnatural amino acid, wherein the unnatural amino acid is biosynthesized in vivo either naturally or through genetic manipulation. The unnatural amino acid is optionally added to a growth medium for at least the first or second organism.

In one embodiment, selecting (e.g., negatively selecting) or screening the library for (optionally mutant) tRNAs that are aminoacylated by an aminoacyl-tRNA synthetase (step (b)) includes: introducing a toxic marker gene, wherein the toxic marker gene comprises at least one of the selector codons (or a gene that leads to the production of a toxic or static agent or a gene essential to the organism wherein such marker gene comprises at least one selector codon) and the library of (optionally mutant) tRNAs into a plurality of cells from the second organism; and, selecting surviving cells, wherein the surviving cells contain the pool of (optionally mutant) tRNAs comprising at least one orthogonal tRNA or nonfunctional tRNA. For example, surviving cells can be selected by using a comparison ratio cell density assay.

In another embodiment, the toxic marker gene can include two or more selector codons. In another embodiment of the methods, the toxic marker gene is a ribonuclease barnase gene, where the ribonuclease bamase gene comprises at least one amber codon. Optionally, the ribonuclease barnase gene can include two or more amber codons.

In one embodiment, selecting or screening the pool of (optionally mutant) tRNAs for members that are aminoacylated by an introduced RS can include: introducing a positive selection or screening marker gene, wherein the positive marker gene comprises a drug resistance gene (e.g., β-lactamase gene, comprising at least one of the selector codons, such as at least one amber stop codon) or a gene essential to the organism, or a gene that leads to detoxification of a toxic agent, along with the RS, and the pool of (optionally mutant) tRNAs into a plurality of cells from the second organism; and, identifying surviving or screened cells grown in the presence of a selection or screening agent, e.g., an antibiotic, thereby providing a pool of cells possessing the at least one recombinant tRNA, where the at least recombinant tRNA is aminoacylated by the RS and inserts an amino acid into a translation product encoded by the positive marker gene, in response to the at least one selector codons. In another embodiment, the concentration of the selection and/or screening agent is varied.

Methods for generating specific tRNA/RS pairs are also provided. Methods include: (a) generating a library of mutant tRNAs derived from at least one tRNA from a first organism; (b) negatively selecting or screening the library for (optionally mutant) tRNAs that are aminoacylated by an aminoacyl-tRNA synthetase (RS) from a second organism in the absence of a RS from the first organism, thereby providing a pool of (optionally mutant) tRNAs; (c) selecting or screening the pool of (optionally mutant) tRNAs for members that are aminoacylated by an introduced RS, thereby providing at least one recombinant tRNA. The at least one recombinant tRNA recognizes a selector codon and is not efficiency recognized by the RS from the second organism and is preferentially aminoacylated by the RS. The method also includes (d) generating a library of (optionally mutant) RSs derived from at least one aminoacyl-tRNA synthetase (RS) from a third organism; (e) selecting or screening the library of mutant RSs for members that preferentially aminoacylate the at least one recombinant tRNA in the presence of an unnatural amino acid and a natural amino acid, thereby providing a pool of active (optionally mutant) RSs; and, (f) negatively selecting or screening the pool for active (optionally mutant) RSs that preferentially aminoacylate the at least one recombinant tRNA in the absence of the unnatural amino acid, thereby providing the at least one specific tRNA/RS pair, wherein the at least one specific tRNA/RS pair comprises at least one recombinant RS that is specific for the unnatural amino acid and the at least one recombinant tRNA. Specific tRNA/RS pairs produced by the methods are included. For example, the specific tRNA/RS pair can include, e.g., a mutRNATyr-mutTyrRS pair, such as a mutRNATyr-SS12TyrRS pair, a mutRNALeu-mutLeuRS pair, a mutRNAThr-mutThrRS pair, a mutRNAGlu-mutGluRS pair, or the like. Additionally, such methods include wherein the first and third organism are the same.

Methods for selecting a tRNA-tRNA synthetase pair for use in an in vivo translation system of a second organism are also included in the present invention. The methods include: introducing a marker gene, a tRNA and an aminoacyl-tRNA synthetase (RS) isolated or derived from a first organism into a first set of cells from the second organism; introducing the marker gene and the tRNA into a duplicate cell set from a second organism; and, selecting for surviving cells in the first set that fail to survive in the duplicate cell set or screening for cells showing a specific screening response that fail to give such response in the duplicate cell set, wherein the first set and the duplicate cell set are grown in the presence of a selection or screening agent, wherein the surviving or screened cells comprise the orthogonal tRNA-tRNA synthetase pair for use in the in the in vivo translation system of the second organism. In one embodiment, comparing and selecting or screening includes an in vivo complementation assay. The concentration of the selection or screening agent can be varied.

In certain embodiments, tRNAs may be used to incorporate unnatural amino acids into a protein. Exemplary unnatural amino acids are described, for example, in U.S. Patent Application Publication No. 2003/0108885 and include, for example, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine. Additionally, other examples optionally include (but are not limited to) an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photo caged amino acid; a photoisomerizable amino acid; a biotin or biotin-analogue containing amino acid; a glycosylated or carbohydrate modified amino acid; a keto containing amino acid; an amino acid comprising polyethylene glycol; an amino acid comprising polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

For example, many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs of the invention include, but are not limited to, α-hydroxy derivatives, β-substituted derivatives, cyclic derivatives, and amide substituted glutamine derivatives. Example phenylalanine analogs include, but are not limited to, meta-substituted phenylalanines, wherein the substituent comprises a hydroxy group, a methoxy group, a methyl group, an allyl group, an acetyl group, or the like. Specific examples of unnatural amino acids include, but are not limited to, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In certain embodiments, unnatural amino acids may be selected or designed to provide additional characteristics unavailable in the twenty natural amino acids. For example, unnatural amino acid are optionally designed or selected to modify the biological properties of a protein, e.g., into which they are incorporated. For example, the following properties are optionally modified by inclusion of an unnatural amino acid into a protein: toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, ability to react with other molecules, e.g., covalently or noncovalently, and the like.

It should be understood, that various methods for producing modified tRNAs and/or tRNA synthetases have been described with reference to unnatural amino acids. However, in certain embodiments, the methods may be applied to producing modified tRNAs and/or tRNA synthetases that recognize non-wild-type amino acids, e.g., tRNAs and/or tRNA synthetases that insert a natural amino acid in response to a codon that is not normally inserted in response to that codon in a wild-type cell not comprising the modified tRNA and/or tRNA synthetase.

7. Exemplary Organisms

The methods described herein may be used to produce modified, partially synthetic, and/or fully synthetic genomes for a variety of cells, including eukaryotic, prokaryotic, diploid, or haploid organisms. The resulting organisms having modified, partially synthetic, and/or fully synthetic genomes may be single cell organisms (e.g., bacteria, e.g., Mycobacterium spp., e.g., M. tuberculosis) or may be derived from multicellular organisms (transgenic organisms, such as insects (e.g., Drosophila), worms (e.g., Caenorhabditis spp, e.g., C. elegans) and higher animals (e.g., transgenic mammals such as mice, rats, rabbits, hamsters, etc.). In certain embodiments, a cell having a modified, partially synthetic, and/or fully synthetic genome is a naturally diploid cell, preferably yeast cells (e.g., Saccharomyces spp. (e.g., S. cerevisiae), Candida spp. (e.g., C. albicans)) or mammalian cells (e.g., mouse, monkey, or human).

In an exemplary embodiment, the methods described herein may be used to produce a modified, partially synthetic, and/or fully synthetic genome for virulent or pathogenic organisms for which it is important to prevent spread of their genetic material in the environment.

Examples of infectious bacteria include, for example, Helicobacter pylori, Borrelia burgdorferi, Legionella pneumophilia, Mycobacterium sps. (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Examples of infectious fungi include, for example, Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium falciparum and Toxoplasma gondii.

Examples of disease causing viruses include, for example, Retroviridae (e.g., human immunodeficiency viruses, such as HIV-1 (also referred to as HTLV-III, LAV or HTLV-III/LAV, See Ratner, L. et al., Nature, Vol. 313, Pp. 227-284 (1985); Wain Hobson, S. et al, Cell, Vol. 40: Pp. 9-17 (1985)); HIV-2 (See Guyader et al., Nature, Vol. 328, Pp. 662-669 (1987); European Patent Publication No. 0 269 520; Chakraborti et al., Nature, Vol. 328, Pp. 543-547 (1987); and European Patent Application No. 0 655 501); and other isolates, such as HIV-LP (International Publication No. WO 94/00562 entitled “A Novel Human Immunodeficiency Virus”; Picornaviridae (e.g., polio viruses, hepatitis A virus, (Gust, I. D., et al., Intervirology, Vol. 20, Pp. 1-7 (1983); entero viruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Any means for the introduction of polynucleotides into eukaryotic or prokaryotic cells may be used in accordance with the compositions and methods described herein. Suitable methods include, for example, direct needle microinjection, transfection, electroporation, retroviruses, adenoviruses, adeno-associated viruses; Herpes viruses, and other viral packaging and delivery systems, polyamidoamine dendrimers, liposomes, and more recently techniques using DNA-coated microprojectiles delivered with a gene gun (called a biolistics device), or narrow-beam lasers (laser-poration). In one embodiment, nucleic acid constructs may be delivered in a complex with a colloidal dispersion system. A colloidal system includes macromolecule complexes, nanocapsules, microspheres, beads, and lipid-based systems including oil-in-water emulsions, micelles, mixed micelles, and liposomes. An exemplary colloidal system of this invention is a lipid-complexed or liposome-formulated DNA. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

In an exemplary embodiment, the methods and compositions described herein may be used in a variety of applications in plants. For example, it may be desirable to produce modified, partially synthetic, and/or fully synthetic plant genome that has been modified for purposes of crop development without concern that such traits may spread beyond a controlled environment. For example, modifications of interest may be reflective of the commercial markets and interests of those involved in the development of a crop, including, for example, genes encoding agronomic traits, insect resistance, disease resistance, herbicide resistance, sterility, grain characteristics, commercial products, genes involved in oil, starch, carbohydrate, or nutrient metabolism, genes affecting kernel size, sucrose loading, and the like, and genes involved in grain quality such as levels and types of oils, saturated and unsaturated, quality and quantity of essential amino acids, and levels of cellulose.

Plants and plant cells may be transformed using any method known in the art. In one embodiment, Agrobacterium is employed to introduce a DNA construct into plants. Such transformation typically uses binary Agrobacterium T-DNA vectors (Bevan, 1984, Nuc. Acid Res. 12:8711-8721), and the co-cultivation procedure (Horsch et al., 1985, Science 227:1229-1231). Generally, the Agrobacterium transformation system is used to engineer dicotyledonous plants (Bevan et al., 1982, Ann. Rev. Genet 16:357-384; Rogers et al., 1986, Methods Enzymol. 118:627-641). The Agrobacterium transformation system may also be used to transform, as well as transfer, DNA to monocotyledonous plants and plant cells. (see Hernalsteen et al., 1984, EMBO J 3:3039-3041; Hooykaas-Van Slogteren et al., 1984, Nature 311:763-764; Grimsley et al., 1987, Nature 325:1677-179; Boulton et al., 1989, Plant Mol. Biol. 12:31-40; and Gould et al., 1991, Plant Physiol. 95:426-434).

In other embodiments, various alternative methods for introducing recombinant nucleic acid constructs into plants and plant cells may also be utilized. These other methods are particularly useful where the target is a monocotyledonous plant or plant cell. Alternative gene transfer and transformation methods include, but are not limited to, particle gun bombardment (biolistics), protoplast transformation through calcium-, polyethylene glycol (PEG)- or electroporation-mediated uptake of naked DNA (see Paszkowski et al., 1984, EMBO J 3:2717-2722, Potrykus et al., 1985, Molec. Gen. Genet. 199:169-177; Fromm et al., 1985, Proc. Nat. Acad. Sci. USA 82:5824-5828; and Shimamoto, 1989, Nature 338:274-276) and electroporation of plant tissues (D'Halluin et al., 1992, Plant Cell 4:1495-1505). Additional methods for plant cell transformation include microinjection, silicon carbide mediated DNA uptake (Kaeppler et al., 1990, Plant Cell Reporter 9:415-418), and microprojectile bombardment (see Klein et al., 1988, Proc. Nat. Acad. Sci. USA 85:4305-4309; and Gordon-Kamm et al., 1990, Plant Cell 2:603-618). In various methods, selectable markers may be used, at least initially, in order to determine whether transformation has actually occurred. Useful selectable markers include enzymes which confer resistance to an antibiotic, such as gentamycin, hygromycin, kanamycin and the like. Alternatively, markers which provide a compound identifiable by a color change, such as GUS, or luminescence, such as luciferase, may be used. For plastid transformation, biolistics according the method of Svab and Maliga (Svab et al., 1993, Proc. Natl. Acad. Sci. USA 90: 913-917) is preferred.

The methods and compositions described herein may be practiced with any plant and/or plant genome. Such plants include but are not limited to, monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis, tobacco).

### EQUIVALENTS

The present invention provides among other things methods for assembling large polynucleotide constructs and organisms having increased genomic stability. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: U.S. Patent Publication Nos. 2005/0009049; 2003/010885; 2003/0082575; 2005/059096; 2005/0136447; 2005/0130205; and 2004/0142452; and Heeb S, et al. Mol Plant Microbe Interact. (2000) 13(2): 232-7; Waters V L Nat Genet. (2001) 29(4): 375-6; Guiney D G, et al. Plasmid. (1988) 20(3): 259-65; Posfai G, et al. Nucleic Acids Res. (1999) 27(22): 4409-15; Li M Z and Elledge S J. Nat Genet. (2005) 37(3): 311-9; Yoon, T G et al., Genetic Analysis: Biomolecular Engineering 14: 89-95 (1998); and Chevalier B S, et al. Mol Cell. (2002) 10(4): 895-905.

What is claimed:

1. A method of reducing or preventing translation of functional transposase in a cell, the method comprising:
i) providing a plurality of cells comprising a plurality of polynucleotide constructs, wherein a portion of the plurality of polynucleotide constructs comprise sequence encoding a first selectable marker and a portion of the plurality of polynucleotide constructs comprise sequence encoding a second selectable marker;
ii) conducting pairwise conjugations by mixing pairs of cells, wherein each pair comprises a cell having at least one polynucleotide construct encoding said first selectable marker and a cell having at least one polynucleotide construct encoding said second selectable marker;
iii) selecting cells comprising at least portions of the polynucleotide constructs from both cells involved in the pairwise mixing that have been assembled in a desired manner by selecting cells comprising one of the first or second selectable markers; and
iv) reiteratively repeating said steps ii) and iii) to form a desired polynucleotide product;
wherein said plurality of polynucleotide constructs together comprise a modification in at least a substantial portion of open reading frames or regulatory regions of transposase genes, and wherein said modification causes a reduction in or prevents translation of functional transposase in a cell.

2. The method of claim 1, wherein at least a portion of said polynucleotide constructs are constructed from synthetic DNA.

3. The method of claim 1, further comprising excising a plurality of polynucleotide sequence segments from a naturally-occurring genome and modifying the sequences of said segments thereby forming said plurality of polynucleotide constructs.

4. A method for introducing a plurality of predetermined nucleotide changes throughout a polynucleotide product, comprising:
modifying one or more nucleotides on each of a plurality of polynucleotide segments from a genome to form a plurality of polynucleotide constructs; and
incorporating said plurality of polynucleotide constructs into said genome thereby introducing a plurality of nucleotide changes throughout said polynucleotide product.

5. The method of claim 4, wherein said polynucleotide product is a genome.

6. The method of claim 4, further comprising excising a plurality of polynucleotide segments from a genome.

7. The method of claim 6, wherein said polynucleotide segments are excised from the genome using site-specific recombination.

8. The method of claim 4, further comprising:
introducing site-specific recombination sequences into the genome at locations flanking one or more polynucleotide segments to be excised from the genome; and
exposing the genome to a site-specific recombinase thereby inducing intramolecular recombination between the site-specific recombination sequences and excising one or more polynucletide segments from the genome.

9. The method of claim 4, wherein said polynucleotide segments are modified by PCR mutagenesis, site-specific mutagenesis, site-specific recombination, or homologous recombination.

10. The method of claim 4, further comprising:
introducing said plurality of polynucleotide constructs into a plurality of cells, wherein the sequences of the plurality of polynucleotide constructs together comprise the sequence of the polynucleotide construct, and wherein each polynucleotide construct comprises sequence encoding at least one of a first or second selectable marker, thereby forming a first set of transfected cells;
mixing pairwise cells from the first set of transfected cells, wherein each pair comprises a cell having polynucleotide construct encoding said first selectable marker and a cell having a polynucleotide construct encoding said second selectable marker, thereby forming a second set of transfected cells;
reiteratively repeating said mixing step, wherein the second set of transfected cells becomes the first set of transfected cells for the next round of pairwise mixing, thereby incorporating said plurality of polynucleotide constructs into said polynucleotide product and introducing a plurality of nucleotide changes throughout said polynucleotide product.

11. The method of claim 10, wherein the pairwise mixing of cells involves conjugation and transfer of a modified polynucleotide segment from one cell to another cell.

12. The method of claim 4, wherein the predetermined nucleotide changes comprise changing all occurrences of at least a first codon in a given sequence to a second codon that is degenerate to the first codon.

13. The method of claim 12, wherein the first and second codons are stop codons.

14. The method of claim 12, wherein the predetermined nucleotide changes comprise changing one or more codons to the first codon wherein the codons that are changed are not degenerate to the first codon.

15. The method of claim 12, wherein the genome is contained in a cell that does not express a wild-type tRNA that recognizes the first codon.

16. The method of claim 15, wherein the cell contains at least one gene encoding a first modified tRNA that recognizes said first codon but is charged with an amino acid not normally encoded by the first codon.

17. The method of claim 13, wherein the genome is contained in a cell that does not express a wild-type release factor that recognizes the first codon.

18. A method of assembling a polynucleotide product comprising:
(a) selecting a double stranded initiating polynucleotide construct;
(b) contacting said initiating polynucleotide construct with a next polynucleotide construct in the presence of a recombination system, wherein said next polynucleotide construct is double stranded and a terminal region of said next polynucleotide construct comprises substantial sequence homology with a terminal region of said initiating polynucleotide construct, and wherein said next polynucleotide construct is joined to said initiating polynucleotide construct by homologous recombination at the terminal regions having substantial sequence homology; and
(c) repeating (b) to sequentially add additional double stranded polynucleotide constructs to the extended initiating polynucleotide construct, whereby said polynucleotide product is synthesized.

19. The method of claim 18, wherein said polynucleotide constructs are introduced into the genome of a cell by homologous recombination.

20. The method of claim 18, wherein the initiating polynucleotide construct and/or the next polynucleotide construct are excised from a genome and optionally modified.


SEQUENCE LISTING (4 sequences)

SEQ ID NO: 1 - DNA, 18 bp, Artificial Sequence
  cleavage site for I-SceI meganuclease
tagggataacagggtaat

SEQ ID NO: 2 - DNA, 25 bp, Artificial Sequence
  cleavage site for I-DmoI meganuclease
gccttgccgggtaagttccggcgcg

SEQ ID NO: 3 - DNA, 24 bp, Artificial Sequence
  cleavage site for I-CreI meganuclease
caaaacgtcgtgagacagtttggt

SEQ ID NO: 4 - DNA, 24 bp, Artificial Sequence
  cleavage site for I-DreI-3 meganuclease
caaaacgtcgtaagttccggcgcg